(19) United States
(12) Patent Application Publication
(10) Pub. No.: US 2025/0325286 A1
(43) Pub. Date: Oct. 23, 2025

(54) Methods and Microneedle Devices for Vaccinations with Enhanced Delivery and Acceptance

(71) Applicant: Micron Biomedical, Inc., Atlanta, GA (US)

(72) Inventors: Devin V. McAllister, Marietta, GA (US); Sebastien Henry, Smyrna, GA (US)

(21) Appl. No.: 19/182,399

(22) Filed: Apr. 17, 2025

Related U.S. Application Data
(60) Provisional application No. 63/634,998, filed on Apr. 17, 2024.
(60) Provisional application No. 63/646,332, filed on May 13, 2024.

(51) Int. Cl. A61B 17/20; A61B 17/00; A61M 37/00

(52) U.S. Cl. CPC A61B 17/205; A61M 37/0015; A61B 2017/00004; A61M 2037/0023; A61M 2037/0046; A61M 2037/0053; A61M 2202/206; A61M 2202/30; A61M 2205/0238

(57) ABSTRACT

Vaccination methods include applying to a subject a microneedle patch having microneedles including a vaccine formulation including one or more infectious disease antigens. Infant and child subjects were found to tolerate application of the patch for a period effective to administer the antigen. The patch may be applied to the skin of a subject for an application period of five minutes or less, and effective to administer the antigen to the subject, wherein at least 50% of the height of the microneedle dissolves into the skin of the subject during the application period. Thermally stable vaccine formulations for the microneedles are provided. The methods may be effective to protect the subject from the infectious disease even if, prior to the applying step, the drug delivery device is transported or stored out of cold chain conditions for up to 14 days.

### CROSS-REFERENCE TO RELATED APPLICATIONS

This application claim priority to and benefit of U.S. Provisional Patent Application No. 63/634,998, filed Apr. 17, 2024, and U.S. Provisional Patent Application No. 63/646,332, filed May 13, 2024, which are incorporated herein by reference in their entirety.

### BACKGROUND

The present application is generally in the field of methods of vaccinating infants, children, and/or adults with microneedles providing efficacy at least as comparable to conventional needle injections and markedly improved tolerability/favorability of the administration route, overcoming trypanophobia.

Improving the immunization of children is a continuing priority for their protection from a wide range of diseases. For one example, substantial progress has been made in reducing the burden of measles and rubella infections. World Health Organization (WHO) statistics indicate that, since 2000, the annual number of measles deaths has fallen by around 83%, and the number of rubella cases annually has fallen by 96%. The WHO therefore remains committed to measles elimination, recommending that all countries consistently achieve at least 95% two-dose measles vaccine coverage nationwide. In countries with ongoing measles transmission, the first dose of the measles vaccine is recommended at nine-months-of-age and the second dose at 15- to 18-months-of-age. Use of combined measles and rubella vaccines (MRV) also support rubella elimination, which requires about 80% population immunity.

However, despite progress in many regions, the coverage achieved through routine immunizations remains considerably below elimination targets. Consequently, periodic supplementary immunization activities (SIA), which target an entire population for vaccination during an intensive campaign period, continue to be necessary. Such approaches tend to improve coverage equity compared to routine immunization; however, SIA are logistically complex and variable in their capacity to reach children who have not received previous measles or other vaccines. Also, vaccine hesitancy in many parts of the world associated with the fear of side effects and low trust in vaccine contribute to less than ideal vaccination coverage.

It therefore would be desirable to provide methods better suited for vaccination by providing simple, convenient, and easy-to-administer dosage presentations.

### BRIEF SUMMARY

In one aspect, a method is provided for vaccinating a child or infant, wherein the method includes: applying to an area of skin of the child or infant a drug delivery device comprising an array of microneedles comprising a vaccine formulation which comprises at least one infectious disease antigen, wherein the applying is effective to administer the infectious disease antigen to the child or infant via the array of microneedles and to protect the child or infant from an infectious disease. The drug delivery device may be a microneedle patch applied to the skin, preferably of an arm or wrist, of the infant or child, and the infant or child tolerates the application of the patch for the period of application of the patch. The period of the application of the patch may be 5 minutes or less, preferably 1 minute or less. The infectious disease may be selected from the group consisting of influenza, COVID-19, measles, diphtheria, tetanus, pertussis, dengue, hepatitis A, hepatitis B, mumps, human papilloma virus (HPV), pneumococcal, meningococcal, rotavirus, polio, varicella, rubella, smallpox, monkeypox, and combinations thereof. Advantageously, the vaccine formulation may be thermostable in the drug delivery device for at least one of 1 month at 37° C., 12 months at 25° C., or 12 months at 5° C., optionally up to 12 months, at 5° C. followed by 14 days at 40° C. Before the drug delivery device is applied to the area of skin of the child or infant, the drug delivery device may have been transported or stored outside of cold chain conditions for more than 12 hours or for at least 14 days.

In another aspect, a method is provided for vaccinating a subject, wherein the method includes: applying to an area of skin of the subject, for an application period of five (5) minutes or less, a drug delivery device comprising an array of dissolvable microneedles comprising a vaccine formulation which comprises at least one infectious disease antigen, wherein: the applying is effective to penetrate the stratum corneum of the skin with the array of dissolvable microneedles and to administer the at least one infectious disease antigen to the subject, and at least 50%, preferably at least 90%, of the height of the dissolvable microneedle dissolves into the skin of the subject. Preferably, the application period is 5 minutes or less, preferably 1 minute or less, or more preferably 10 seconds or less. The applying is effective to protect the subject from at least one infectious disease corresponding with the at least one infectious disease antigen. In some embodiments, the dissolution alters the geometry of the tip of each microneedles such that it is no longer able to penetrate the stratum corneum of skin.

In yet another aspect, a drug delivery device is provided, which may be useful in the foregoing methods. The drug delivery device has an array of microneedles that include or consist of a vaccine formulation which comprises at least one infectious disease antigen. In a particular embodiment, the vaccine formulation further comprises gelatin, sorbitol, and one or more buffers. In one example, the at least one infectious disease antigen comprises a measles antigen and a rubella antigen.

### BRIEF DESCRIPTION OF THE DRAWINGS

FIG. 1 is a side view of a plurality of microneedles comprising a vaccine composition, according to one or more embodiments of the present disclosure. The microneedle array 10 includes a base substrate 12 with a plurality of microneedles 14.

FIG. 2A is a series of schematics of a microneedle patch, microneedles dissolving into the skin, and removal of the patch (minus the microneedles).

FIG. 2B are microphotographs showing progression of microneedle dissolution in porcine skin.

FIG. 3A is a flow chart depicting a trial profile for a toddler cohort.

FIG. 3B is a flow chart depicting a trial profile for an infant cohort.

FIG. 3C is a flow chart depicting a trial profile for an adult cohort.

FIG. 4A is a perspective view of a microneedle patch for administering a vaccine composition, according to an embodiment of the present disclosure.

FIG. 4B is a schematic depicting microneedle patch of FIG. 4A and its force feedback indicator (FFI). Left: The microneedle patch is shown in its protective cap before use (the green colored ring of the FFI is visible); Middle: the microneedle patch is removed from its protective cap for use and a force (represented by the arrow) is applied to the microneedle patch. The user receives tactile and audible feedback when a minimum threshold force is applied; Right: After the minimum threshold force has been applied, the green colored ring of the FFI is no longer visible.

FIG. 5A is a bar graph showing local solicited adverse events in the toddler cohort.

FIG. 5B is a bar graph showing local solicited adverse events in the infant cohort.

FIG. 6A is a plot of unsolicited adverse events in the toddler cohort with 95% confidence intervals.

FIG. 6B is a plot of unsolicited adverse events in the infant cohort with 95% confidence intervals.

FIG. 6C is a plot of unsolicited adverse events in the adult cohort with 95% confidence intervals.

FIG. 7A is a bar graph showing serum neutralizing antibody seroprotection levels and geometric mean antibody concentrations in the toddler cohort for measles and rubella, with 95% confidence intervals.

FIG. 7B is a bar graph showing serum neutralizing antibody seroprotection levels and geometric mean antibody concentrations in the infant cohort for measles and rubella, with 95% confidence intervals.

FIG. 7C is a bar graph showing serum neutralizing antibody seroprotection levels and geometric mean antibody concentrations in the adult cohort for measles and rubella, with 95% confidence intervals.

FIG. 7D is a line graph showing the reverse cumulative distribution curves for measles and rubella in the toddler cohort.

FIG. 7E is a line graph showing the reverse cumulative distribution curves for measles and rubella in the infant cohort.

FIG. 7F is a line graph showing the reverse cumulative distribution curves for measles and rubella in the adult cohort.

FIG. 8 is a bar graph showing the average percent of microneedle height dissolution among microneedle patches administered to the infant, toddler, and adult cohorts.

FIG. 9A is a bar graph depicting stability data from MRV-MicroNeedle Patches (MNPs) sampled from a clinical batch and stored at 5° C.±3° C. for up to 12 months, demonstrating that the measles and rubella vaccines remained stable for at least 12 months. Note: In FIGS. 9A-9D, the term measles rubella microarray patch (MR MAP) describes the MRV-MNPs.

FIG. 9B is a bar graph depicting stability data from MRV-MNPs sampled from a clinical batch and stored at 2-8° C. (left) and 25° C. and 65% relative humidity (right) for up to 12 months, demonstrating that the measles vaccine remained stable for at least 12 months.

FIG. 9C is a bar graph depicting stability data from MRV-MNPs sampled from a clinical batch and stored at 2-8° C. (left) and 25° C. and 65% relative humidity (right) for up to 12 months, demonstrating that the rubella vaccine remained stable for at least 12 months.

FIG. 9D is a bar graph depicting stability data from MRV-MNPs sampled from a clinical batch and stored at 37° C. for up to 1 month, demonstrating that the measles vaccine remained stable for at least 1 month.

FIG. 9E is a bar graph depicting stability data from MRV-MNPs sampled from a clinical batch and stored at 37° C. for up to 1 month, demonstrating that the rubella vaccine remained stable for at least 1 month.

FIG. 9F is a plot depicting stability data from MRV-MNPs sampled from a clinical batch and stored under Controlled Temperature Chain (CTC) conditions (i.e., at 2-8° C. for 12 months, then stored at 40° C. and 75% relative humidity), demonstrating that the measles vaccine remained stable at 40° C. and 75% relative humidity.

FIG. 9G is a plot depicting stability data from MRV-MNPs sampled from a clinical batch and stored under Controlled Temperature Chain (CTC) conditions (i.e., at 2-8° C. for 12 months, then stored at 40° C. and 75% relative humidity), demonstrating that the rubella vaccine remained stable at 40° C. and 75% relative humidity.

FIG. 9H is a bar graph demonstrating that measles and rubella vaccines remained stable after being subjected to at least 9 freeze-thaw cycles (i.e., −20° C. for 1 day, followed by 25° C. for 1 day).

FIG. 10A is a bar graph depicting the results of a survey directed to parents of the infant participants in the trial asking which of the two methods (MNP or subcutaneous (SC) injection) they believed would be better for administering vaccines to children.

FIG. 10B is a bar graph depicting the results of a survey directed to parents of the toddler participants in the trial asking which of the two methods (MNP or SC injection) they believed would be better for administering vaccines to children.

FIG. 10C is a bar graph depicting the results of a survey directed to adult participants in the trial asking which of the two methods (MNP or SC injection) they believed would be better for administering vaccines to children.

FIG. 10D is a bar graph depicting the results of a survey directed to parents of the infant participants in the trial asking about the good things about vaccinating a child with a MNP.

FIG. 10E is a bar graph depicting the results of a survey directed to parents of the toddler participants in the trial asking about the good things about vaccinating a child with a MNP.

FIG. 10F is a bar graph depicting the results of a survey directed to the adult participants in the trial asking about the good things about vaccinating a child with a MNP.

FIG. 10G is a pie chart depicting the results of a survey directed to parents of the infant participants in the trial asking about the bad things about vaccinating a child with a MNP.

FIG. 10H is a pie chart depicting the results of a survey directed to parents of the toddler participants in the trial asking about the bad things about vaccinating a child with a MNP.

FIG. 10I is a pie chart depicting the results of a survey directed to the adult participants in the trial asking about the bad things about vaccinating a child with a MNP.

FIG. 10J is a bar graph depicting the results of a survey directed to parents of the infant participants in the trial asking whether they would be willing to have new volunteers administer to their child a MNP vaccine (top) as compared to a vaccine administered by SC injection (bottom).

FIG. 10K is a bar graph depicting reported crying in infants (top) and toddlers (bottom) prior to and during vaccination by MNP (left) and SC injection (right).

FIGS. 11A and 11B are graphs depicting stability data from MRV-MNPs sampled from a clinical batch and stored at 2-8° C. (left) and 25° C. (right) and 65% relative humidity for up to 24 months, demonstrating that the measles vaccine remained stable. (Note: In FIGS. 11A-9D, the term measles rubella microarray patch (MR MAP) describes the MRV-MNPs)

FIGS. 11C and 11D are graphs depicting stability data from MRV-MNPs sampled from a clinical batch and stored at 2-8° C. (left) and 25° C. (right) and 65% relative humidity for up to 24 months, demonstrating that the rubella vaccine remained stable.

FIG. 11E is a plot depicting stability data from MRV-MNPs sampled from a clinical batch and stored under Controlled Temperature Chain (CTC) conditions (i.e., at 2-8° C. for 24 months, then stored at 40° C. and 75% relative humidity), demonstrating that the measles vaccine remained stable at 40° C. and 75% relative humidity.

FIG. 11F is a plot depicting stability data from MRV-MNPs sampled from a clinical batch and stored under Controlled Temperature Chain (CTC) conditions (i.e., at 2-8° C. for 24 months, then stored at 40° C. and 75% relative humidity), demonstrating that the rubella vaccine remained stable at 40° C. and 75% relative humidity.

### DETAILED DESCRIPTION

Methods of administering a vaccine using microneedle patches have been demonstrated with notable results. Microneedle patches (MNP) offer a number of pragmatic advantages over needle and syringe-based vaccination administration, and can provide similar or enhanced immunogenicity against infectious diseases, such as measles and rubella. The methods and compositions disclosed herein provide microneedle patches that achieve equivalent or improved tolerability, safety, stability, immunogenicity, and acceptability compared with conventional vaccination in humans. The term “microneedle” may be referred to herein by the abbreviation “MN”.

In some embodiments, the method includes administering a single dose of vaccine into a biological tissue of an infant or child using a microneedle patch applied to the skin of the child or infant. The single dose may be a first or second (or subsequent) dose administered to the infant or child. The infant or child can be any age. For example, The infant may be a newborn four to eighteen months old, or at least four months old. The child may be four to six years old.

In some embodiments, such as in some developing countries, the first dose of a measles rubella vaccine may be given to 9-10 month old infants, with the second dose given to 15-18 month old toddlers. In some embodiments, the first dose of a measles rubella vaccine may be given to infants as young as 6 months old. In some other embodiments, such as in the U.S., where the CDC recommends all children get two doses of MMR (measles-mumps-rubella) vaccine, the first dose may be administered to infants at ages 12 to 15 months, with the second dose administered to children at ages 4 to 6 years. The ages for other vaccines and for other regions/countries may differ.

The microneedle patch may be applied anywhere on the skin of the child or infant, but is preferably, in at least some embodiments, applied to the arm or wrist of the infant or child. In some embodiments, the microneedle patch is applied manually, optionally with the an active application tool incorporated into the patch or as a separate apparatus. For example, the microneedle patch can be applied with the aid of a force feedback indicator (FFI) or, alternatively, with the aid of a separate applicator. The FFI may provide audible, tactile, and/or visual feedback indicating that the patch has been properly applied to the skin (i.e., such that enough force was applied by the user to enable the microneedles to be inserted effectively). The microneedle patches may be applied by a medical or healthcare professional with prior training and experience in using needles and syringes or someone with little to no medical or healthcare experience with no or little training and experience in using needles and syringes. In some instances, the microneedle patches may be self-administered or applied by a parent or family member to a child or other family or community member. In some embodiments, the microneedle patches can be mailed to the patient's home and administered.

In a preferred embodiment, the microneedle patch includes an array of dissolvable microneedles that include one or more vaccine formulations and extend from a base. The vaccine formulation(s) include(s) one or more infectious disease antigen(s) that protect(s) the child or the infant (or an adult) from one or more infectious disease(s) including, but not limited to (e.g., in the case cross-protective vaccines), those corresponding with the infectious disease antigen(s) administered. For example, a microneedle patch may include antigens for measles, mumps, and rubella (MMR) or for measles and rubella (MR). In other examples, a microneedle patch may include a cross-protective vaccine. A cross-protective vaccines can be designed to provide immunity against multiple pathogens or strains, even though they contain antigens from only one or a few of those pathogens because the immune response generated by the vaccine can recognize and respond to similar antigens present in different pathogens.

In some preferred embodiments, at least 50% (for example, at least 75% or at least 90%), of the height of the microneedles may dissolve into the skin, such that dissolution of each microneedle is effective to change the shape of the microneedle so that it becomes dull and cannot penetrate another biological tissue. In some other embodiments, this result may occur with less than 50% of the height of the microneedle dissolved, e.g., between 25% and 50% of the microneedle height (including the distal tip of the microneedle). Accordingly, with such embodiments, there are no sharps waste, risk of needlestick injury or risk of accidental or intentional re-use of the microneedles.

In other preferred embodiments, at least 50% of the height of the microneedle may dissolve into the skin, such that dissolution of each microneedle is effective to administer the infectious disease antigen and protect the subject from the infectious disease, including, but not limited to those corresponding with the infectious disease antigen(s) administered. In some embodiments, between 50% and 100% of the height of the microneedle dissolves, preferably between 60% and 100%, between 60% and 90%, between 60% and 80%, between 60% and 70%, between 70% and 100%, between 70% and 90%, between 70% and 80%, between 80% and 100%, between 80% and 90%, or between 90% and 100%.

In some cases, the total height of the microneedle is composed a “dissolving” distal portion and a proximal portion that is not a “dissolving” a portion. The “dissolving” portion of the microneedle can be formulated with water soluble excipients. The presence and height of a non-dissolving proximal portion can reduce the % of the total microneedle height that dissolves.

In some embodiments, the subject is an infant. In one particular embodiment the infant may be between newborn to eighteen months old. Between about 80% and 90% of the height of the microneedle may be dissolved into the skin of the infant, and more preferably, at least 98% of the height of the microneedle is dissolved. In some embodiments, the subject is a toddler. Between about 80% and 90% of the height of the microneedle may be dissolved into the skin of the toddler, and more preferably, at least 91% of the height of the microneedle is dissolved. In some embodiments, the subject is a child. In one particular embodiment, the subject may be between four and six years old. Between about 75% and 95% of the height of the microneedle may be dissolved into the skin of the child. In some embodiments, the subject is an adult. In one particular embodiment, the adult may be between eighteen and forty years old. Between about 60% and 90% of the height of the microneedle may be dissolved into the skin of the adult, more preferably, at least 82% of the height of the microneedle is dissolved.

In some embodiments, the microneedle patch is applied to a tissue of the subject. (It is understood that following a period of being applied, the microneedle patch (e.g., it backing and other parts remaining after the administration of the vaccine formulation) is removed from the tissue.) The tissue can be mucosal or skin tissue. In one particular embodiment, the microneedle patch is applied to the skin, preferably of an arm or wrist, and is tolerated by the subject for the period of application of the patch. The period of application of the patch may be 5 minutes or less, preferably 1 minute or less, or more preferably, 30 seconds or less or 10 seconds or less.

In some embodiments, the protection of the subject from an infectious disease is demonstrated by a seroconversion of the subject to the infectious disease antigen delivered by the microneedles that is statistically equivalent to or greater than the seroconversion of the infectious disease antigen delivered by subcutaneous or intramuscular injection. That is, the same seroconversion/antibody titers may be induced by the same amount/dose of infectious disease antigen. The protection of the subject from the infectious disease may be demonstrated by a serum neutralizing antibody seroprotection of the subject to the infectious disease antigen that is statistically equivalent to the serum neutralizing antibody seroprotection of the infectious disease antigen delivered by subcutaneous injection at 42 days post vaccination. In some embodiments, the seroprotection rate is statistically equivalent for at least 180 days post vaccination.

In some other embodiments, the protection of the subject from an infectious disease is demonstrated by the level of antibody titers in the subject to the infectious disease antigen delivered by the microneedles that is statistically equivalent to or greater than the level of antibody titers of the infectious disease antigen delivered by subcutaneous or intramuscular injection.

The infectious disease may be a bacterial or viral disease. Non-limiting examples include influenza, COVID-19, measles, diphtheria, tetanus, pertussis, dengue, hepatitis A, hepatitis B, mumps, human papillomavirus, pneumococcal, meningococcal, rabies, rotavirus, polio, varicella, rubella, RSV, zoster/shingles, Hib, dengue, typhoid, anthrax, cholera, Japanese encephalitis, rabies, tuberculosis, and/or yellow fever. The infectious disease antigen of the microneedles may be a conventional antigen or one specifically tailored for use in a dissolvable microneedle.

As used herein, the term “antigen” refers to a substance that is recognized by a subject's immune system and triggers an immune response. Antigens can include proteins or polysaccharides found on the surface of pathogens such as bacteria, viruses, and fungi. They can also be toxins, foreign particles, or cells from another organism. The immune system identifies these antigens as foreign and mounts a response to neutralize or eliminate them. Antigens of the present invention can also include a nucleic acid sequence that encodes the antigenic protein. This nucleic acid sequence may be DNA or RNA and can be designed to express the antigenic protein within a host organism, thereby eliciting an immune response.

In some embodiments, the array of microneedles includes a plurality of dissolvable microneedles where the vaccine formulation is contained within the microneedles. For example, the microneedles may be formed of a water-soluble matrix material in which the infectious disease antigen or other prophylactic or therapeutic agent is dispersed, such as described in U.S. Pat. No. 10,828,478, which is incorporated herein by reference. In other embodiments, the microneedles are coated with a coating having the vaccine formulation dispersed therein.

The array of microneedles may be applied to the skin for any suitable period of time, and preferably the period is no longer than necessary to ensure effective separation of at least the drug-containing portion (e.g., the vaccine-containing portion) of the microneedles from the base of the array. In some embodiments, the period of the application of the patch is 5 minutes or less, preferably 1 minute or less, or more preferably 10 seconds or less. The separated vaccine-containing portion may dissolve fully over such short periods. In various embodiments, the patch is applied for ten seconds, 30 seconds, 45 seconds, one minute, two minutes, three minutes, four minutes, or five minutes. It has been discovered that microneedle application is well tolerated by infants and children. In one preferred embodiment, the microneedle patch is applied for five minutes or less.

In another preferred embodiment, the patch is applied for one minute. The World Health Organization has recently drawn attention to the fact that the introduction of microneedle patches into immunization campaigns brings new considerations for ensuring proper administration and adherence to usage guidelines. Depending on the specific design and technology, microneedle patches can be worn for times ranging from 10 seconds to twenty minutes. This introduces logistical challenges, such as determining how to ensure the patch is worn for the required duration, identifying who supervises the subject to prevent early removal, who is responsible for timing the process, and who ultimately removes the patch. Thus, microneedle patch wear times of one minute or less, such as those disclosed in the present invention, are optimal to allow for efficient immunization campaigns.

In some other (less preferable) embodiments, the microneedle patch may be configured to remain applied to the skin for extended period of hours or days, for example, to allow a vaccine to be released over extended period. In yet another embodiment, the vaccine-containing portion of the microneedles separates quickly from the patch as noted above; however the separated portions may be configured (e.g., with a bioerodible matrix material) to release the vaccine over an extended period. In some embodiments, a microneedle patch may include two or more types of separable microneedles, some dissolving and releasing the vaccine in less than 5 minutes and some configured to delay or extend release of the vaccine for days, weeks, or months.

In some instances, the child or infant may develop a mild local reaction or a mild or moderate systemic reaction to the vaccine within minutes, hours or days of administration. Typically, reactogenicity (i.e., local and/or systemic reactions caused by the vaccine) will occur within 14 days but some of the events may last longer. Some adverse events may even have an onset beyond that period. The local or systemic reaction may indicate that the vaccine was successfully administered. The local or systemic reaction may be mild. Reactogenicity at the site of microneedle patch application may advantageously be used to determine whether a child (or other recipient) has received a vaccine. For example, the mild, local reaction may include an induration response. The induration response in the child or infant occurs earlier in children or infants with previous exposure to the infectious disease antigen than in children or infants without previous exposure to the infectious disease antigen

Tables 1-2 below depict a scaling system that can be used for local and systemic event grading (based on the National Institute of Health, Divisions of AIDS (DAIDS) Table for Grading the Severity of Adult and Pediatric Adverse Events-Corrected Version 2.1 Jul. 2017). In some embodiments of the presently disclosed methods, the vaccine delivered by a microneedle patch as disclosed herein may produce an adverse reaction. The vaccinated individual may be solicited to determine the extent of their reaction. The reaction may be mild and local. The reaction may be mild and systemic. Alternatively, the reaction may be moderate and systemic. The tables below provide context for the reactions characterized in the present disclosure.

### Table 1. Local Adverse Event Grading

TABLE 1A
Adult Cohort: Local Solicited Adverse Event Grading

| | | | | | Grade 4§ |
|---|---|---|---|---|---|
| Local administration | | Grade 1 | Grade 2 | Grade 3 | Potentially Life |
| site | Grade 0 | Mild | Moderate | Severe | Threatening |
| Pain | No pain | Pain causing no or | Pain causing | Pain causing | Pain causing |
| | | minimal limitation to | greater than minimal | inability to perform | inability to perform |
| | | use of limb | limitation to use of | usual social and | basic self-care |
| | | | limb | functional activities | function or |
| | | | | | hospitalization |
| | | | | | indicated |
| Redness/Erythema‡ | <2.5 cm in | 2.5 cm to <5 cm in | ≥5 cm to <10 cm in | ≥10 cm in diameter | Potentially life- |
| (size as well as grade | diameter | diameter OR 6.25 to | diameter OR ≥25 | OR ≥100 cm² | threatening |
| will be collected) | | <25 cm² surface | to <100 cm² surface | surface area OR | consequences (e.g., |
| | | area AND | area OR symptoms | ulceration OR | abscess, exfoliative |
| | | symptoms causing | causing greater than | secondary infection | dermatitis, necrosis |
| | | no or minimal | minimal interference | OR phlebitis OR | involving dermis or |
| | | interference with | with usual social | sterile abscess OR | deeper tissues) |
| | | usual social and | and functional | drainage OR | |
| | | functional activities | activities | symptoms causing | |
| | | | | inability to perform | |
| | | | | usual social and | |
| | | | | functional activities | |
| Swelling/induraton‡ | <2.5 cm in | 2.5 cm to <5 cm in | ≥5 cm to <10 cm in | ≥10 cm in diameter | Potentially life- |
| (size as well as grade | diameter | diameter OR 6.25 | diameter OR ≥25 | OR ≥100 cm² | threatening |
| will be collected) | | to <25 cm² surface | to <100 cm² surface | surface area OR | consequences (e.g. |
| | | area AND | area OR symptoms | ulceration OR | abscess, exfoliative |
| | | symptoms causing | causing greater than | secondary infection | dermatitis, necrosis |
| | | no or minimal | minimal interference | OR phlebitis OR | involving dermis or |
| | | interference with | with usual social | sterile abscess OR | deeper tissues) |
| | | usual social and | and functional | drainage OR | |
| | | functional activities | activities | symptoms causing | |
| | | | | inability to perform | |
| | | | | usual social and | |
| | | | | functional activities | |
| Pruritis (localized | No pruritis | Itching localized to | Itching beyond | Generalized itching | N/A |
| to injection site | at injection | injection site AND | injection site that is | that causes inability | |
| | site | no or minimal | not generalized OR | to perform usual | |
| | | limitation to function | itching that causes | social and functional | |
| | | | more than minimal | activities | |
| | | | loss of function | | |

§Any AE resulting in death will be defined as grade 5 severity.
‡Grading based on the greatest single diameter or measured surface area.

TABLE 1B
Toddler and Infant Cohort: Local and Solicited Adverse Event Grading

| | | | | | Grade 4§ |
|---|---|---|---|---|---|
| Local administration | | Grade 1 | Grade 2 | Grade 3 | Potentially Life |
| site | Grade 0 | Mild | Moderate | Severe | Threatening |
| Pain | No pain | Pain causing no or | Pain causing | Pain causing | Pain causing |
| | | minimal limitation to | greater than | inability to perform | inability to perform |
| | | use of limb | minimal limitation to | usual social and | basic self-care |
| | | | use of limb | functional activities | function or |
| | | | | | hospitalization |
| | | | | | indicated |
| Redness/Erythema‡ | No | ≤2.5 cm in diameter | >2.5 cm in diameter | ≥50% of the | Potentially life- |
| (size as well as grade | redness/ | | with <50% of the | surface area of the | threatening |
| will be collected) | erythema | | surface area of the | extremity segment | consequences (e.g. |
| | | | extremity segment | involved (e.g. lower | abscess, exfoliative |
| | | | involved (e.g. lower | arm or thigh) OR | dermatitis, necrosis |
| | | | arm or thigh) | ulceration OR | involving dermis or |
| | | | | secondary infection | deeper tissues) |
| | | | | OR phlebitis OR | |
| | | | | sterile abscess OR | |
| | | | | drainage | |
| Swelling/induration‡ | No | ≤2.5 cm in diameter | >2.5 cm in diameter | ≥50% of the | Potentially life- |
| (size as well as grade | swelling/ | | with <50% of the | surface area of the | threatening |
| will be collected) | induration | | surface area of the | extremity segment | consequences (e.g. |
| | | | extremity segment | involved (e.g. lower | abscess, exfoliative |
| | | | involved (e.g. lower | arm or thigh) OR | dermatitis, necrosis |
| | | | arm or thigh) | ulceration OR | involving dermis or |
| | | | | secondary infection | deeper tissues) |
| | | | | OR phlebitis OR | |
| | | | | sterile abscess OR | |
| | | | | drainage | |

§Any AE resulting in death will be defined as grade 5 severity.
‡Grading based on the greatest single diameter or measured surface area.

### Table 2. Systemic Adverse Event Grading

TABLE 2A
Adult Cohort: Systemic Solicited Adverse Event Grading

| | | | | | Grade 4§ |
|---|---|---|---|---|---|
| | | Grade 1 | Grade 2 | Grade 3 | Potentially Life |
| Systemic | Grade 0 | Mild | Moderate | Severe | Threatening |
| Acute allergic | No acute | Localized urticaria | Localized urticaria | Generalized urticaria | Anaphylaxis OR life- |
| reactions‡ | allergic | (wheals) with no | (wheals) with | OR angioedema with | threatening |
| | reaction | intervention | intervention indicated | intervention indicated | bronchospasm |
| | | indicated† | OR mild angioedema | OR symptoms of | (wheeze) or |
| | | | with no intervention | mild bronchospasm | laryngeal oedema |
| | | | indicated† | (wheeze) | (stridor) |
| Axillary | <37.5° C. | 37.5 to 38.4° C. | 38.5-38.9° C. | 39.0-40.0° C. | >40° C. |
| Temperature | | | | | |
| Vomiting | No | Transient or | Frequent episodes | Persistent vomiting | Life threatening |
| | vomiting | intermittent AND no | with no or mild | resulting in | consequences (e.g., |
| | | or minimal | dehydration - oral | orthostatic | hypotensive shock) |
| | | interference with oral | rehydration solution | hypotension OR | |
| | | intake | indicated | aggressive | |
| | | | | rehydration indicated | |
| | | | | (e.g. intravenous | |
| | | | | fluids) | |
| Diarrhoea | No | Transient or | Persistent episodes | Increase of ≥7 | Life threatening |
| | diarrhoea | intermittent episodes | of unformed watery | stools per 24-hour | consequences (e.g. |
| | | of unformed stool OR | stool OR increase of | period OR | hypotensive shock) |
| | | increase of ≤3 stools | 4 to 6 stools over | intravenous fluid | |
| | | over baseline per 24- | baseline per 24-hour | replacement | |
| | | hour period | period | indicated | |
| Headache | No | Symptoms causing | Symptoms causing | Symptoms causing | Symptoms causing |
| | headache | no or minimal | greater than minimal | inability to perform | inability to perform |
| | | interference with | interference with | usual social and | basic self-care |
| | | usual social and | usual social and | functional activities | functions OR |
| | | functional activities | functional activities | | hospitalization |
| | | | | | indicated OR |
| | | | | | headache with |
| | | | | | significant |
| | | | | | impairment of |
| | | | | | alertness or other |
| | | | | | neurological function |
| Fatigue | No | Fatigue causing no | Fatigue causing | Fatigue causing | Incapacitating |
| | fatigue | or minimal | greater than minimal | inability to perform | symptoms of fatigue |
| | | interference with | interference with | usual social and | causing inability to |
| | | usual social and | usual social and | functional activities | preform basic self- |
| | | functional activities | functional activities | | care functions |
| Myalgia | No | Muscle pain causing | Muscle pain causing | Muscle pain causing | Incapacitating |
| | muscle | no or minimal | greater than minimal | inability to perform | muscle pain causing |
| | pain | interference with | interference with | usual social and | inability to preform |
| | | usual social and | usual social and | functional activities | basic self-care |
| | | functional activities | functional activities | | functions |
| Arthralgia | No joint | Join pain causing no | Joint pain causing | Joint pain causing | Incapacitating joint |
| | pain | or minimal | greater than minimal | inability to perform | pain causing inability |
| | | interference with | interference with | usual social and | to preform basic self- |
| | | usual social and | usual social and | functional activities | care functions |
| | | functional activities | functional activities | | |
| Rash at a site | No rash | Localized rash at a | Diffuse rash OR | Diffuse rash AND | Extensive of |
| other than | | site other than the | target lesions | vesicles or limited | generalized bullous |
| product | | product | | number of bullae or | lesions OR |
| administration | | administration site. | | superficial ulceration | ulceration of mucous |
| site | | | | of mucous | membranes involving |
| | | | | membranes at one | tow or more distinct |
| | | | | site | mucosal sites OR |
| | | | | | Stevens-Johnson |
| | | | | | syndrome OR toxic |
| | | | | | epidermal necrolysis |

For all solicited systemic AE the terminology used in local languages will be standardized in advance and age appropriate social and functional activities will be defined.
§Any AE resulting in death will be defined as grade 5 severity.
†Localized in this case does not need to reflect urticaria localized to the site of the injection itself but rather urticarial localized to one anatomical location.

TABLE 2B
Toddler and Infant Cohort: Systemic Solicited Adverse Event Grading

| | | | | | Grade 4§ |
|---|---|---|---|---|---|
| | | Grade 1 | Grade 2 | Grade 3 | Potentially Life |
| Systemic | Grade 0 | Mild | Moderate | Severe | Threatening |
| Acute allergic | No acute | Localized urticaria | Localized urticaria | Generalized urticaria | Anaphylaxis OR life- |
| reactions‡ | allergic | (wheals) with no | (wheals) with | OR angioedema with | threatening |
| | reaction | intervention | intervention | intervention | bronchospasm |
| | | indicated† | indicated OR mild | indicated OR | (wheeze) or |
| | | | angioedema with no | symptoms of mild | laryngeal oedema |
| | | | intervention | bronchospasm | (stridor) |
| | | | indicated† | (wheeze) | |
| Axillary | <37.5° C. | 37.5 to 38.4° C. | 38.5-38.9° C. | 39.0-40.0° C. | >40° C. |
| Temperature | | | | | |
| Vomiting | No | Transient or | Frequent episodes | Persistent vomiting | Life threatening |
| | vomiting | intermittent AND no | with no or mild | resulting in | consequences (e.g. |
| | | or minimal | dehydration - oral | orthostatic | hypotensive shock) |
| | | interference with oral | rehydration solution | hypotension OR | |
| | | intake | indicated | aggressive | |
| | | | | rehydration indicated | |
| | | | | (e.g. intravenous | |
| | | | | fluids) | |
| Diarrhoea | No | Liquid stools (less | Liquid stools with | Liquid stool with | Life threatening |
| | diarrhoea | formed than usual) | increased number of | moderate | consequences (e.g. |
| | | but usual number of | stools OR mild | dehydration | liquid stool resulting |
| | | stools | dehydration | | in severe |
| | | | | | dehydration, |
| | | | | | hypotensive shock) |
| Irritability | No | Crying more than | Crying more than | Crying more than | Requiring |
| | irritability | normal/irritability but | normal/irritability | normal/irritability | hospitalization due |
| | | no or minimal | causing greater than | preventing usual | to irritability |
| | | interference with | minimal interference | social and functional | |
| | | usual social and | with usual social and | activities | |
| | | functional activities | functional activities | | |
| Drowsiness | No | Sleeping more than | Sleeping more than | Sleeping more than | Requiring |
| | drowsiness | normal/drowsiness | normal/drowsiness | normal/drowsiness | hospitalization due |
| | | but no or minimal | causing greater than | preventing usual | to drowsiness |
| | | interference with | minimal interference | social and functional | |
| | | usual social and | with usual social and | activities | |
| | | functional activities | functional activities | | |
| Appetite | Eating/ | Eating/feeding less | Eating/feeding less | Eating/feeding less | Requiring |
| | feeding | than normal but no | than normal with | than normal | hospitalization due |
| | normally | or minimal | greater than minimal | preventing usual | to not eating/feeding. |
| | | interference with | interference with | social and functional | |
| | | usual social and | usual social and | activities | |
| | | functional activities | functional activities | | |
| Rash at a site | No rash | Localized rash at a | Diffuse rash OR | Diffuse rash AND | Extensive of |
| other than | | site other than the | target lesions | vesicles or limited | generalized bullous |
| product | | product | | number of bullae or | lesions OR |
| administration | | administration site. | | superficial ulceration | ulceration of mucous |
| site | | | | of mucous | membranes |
| | | | | membranes at one | involving tow or |
| | | | | site | more distinct |
| | | | | | mucosal sites OR |
| | | | | | Stevens-Johnson |
| | | | | | syndrome OR toxic |
| | | | | | epidermal necrolysis |

For all solicited systemic AE the terminology used in local languages will be standardized in advance and age appropriate social and functional activities will be defined.
§Any AE resulting in death will be defined as grade 5 severity.
†Localized in this case does not need to reflect urticaria localized to the site of the injection itself but rather urticarial localized to one anatomical location.

Table 3 below depicts a scaling system that can be used for local and systemic event grading (based on the Cancer Therapy Evaluation Program, Common Terminology Criteria for AEs, Version 3.0, DCTD, NCI, NIH, DHHS Mar. 31, 2003, published Aug. 9, 2006). In some embodiments of the presently disclosed methods, the vaccine delivered by a microneedle patch as disclosed herein may produce a reaction. The vaccinated individual may be solicited to determine the extent of their reaction. The reaction may be mild and local. The reaction may be mild and systemic. Alternatively, the reaction may be moderate and systemic. The tables herein provide context for the reactions characterized in the Example section of the present application.

In some embodiments of the presently disclosed invention, the vaccine delivered by a microneedle patch may be believed to produce a reaction, and the vaccinated individual may volunteer to share information about that reaction that may nor not be related to the vaccine (i.e., an unsolicited event). Examples of severity grading for unsolicited adverse events are included, for example, in Table 3 below.

TABLE 3
Other adverse events can be graded for severity according
to the criteria set out in the Division of AIDS (DAIDS)
Table for Grading the Severity of Adult and Pediatric AEs
(V2.1 July 2017) or as described in the table below:

| | | Description |
|---|---|---|
| Grade 1 | Mild | No interference with activity and no or minimal |
| | | intervention¹ required |
| Grade 2 | Moderate | Some interference with activity or requires more |
| | | than minimal intervention |
| Grade 3 | Severe | Prevents daily activity and required significant |
| | | medical intervention |
| Grade 4 | Life- | Life threatening consequences requiring urgent |
| | threatening | medical intervention |
| Grade 5 | Death | Results in death |

¹e.g., one or two doses of antipyretic or simple analgesic medication or local topical treatment.

The microneedle patch may be as or more effective in administering a vaccine to children or infants as with other routes of administration (e.g., subcutaneous or intramuscular injection, nasal administration, or oral administration). The level of protection against the infectious disease provided by the vaccine may be demonstrated by a seroconversion rate or a serum neutralizing antibody seroprotection rate to the infectious disease antigen. In some embodiments, the methods provided herein provide a seroprotection rate that is statistically equivalent to, or greater than, the seroconversion rate for vaccines administered by subcutaneous injection (or intramuscular injection or other routes of administration). The duration may be measured at different time points, depending on the particular vaccine and/or regulatory authority. For example, it may be at least 28 days or at least 42 days post-vaccination for influenza, or 42 days post-vaccination for measles/rubella.

The currently disclosed methods of vaccination with microneedle patches may be effective to improve (e.g., reduce) or overcome trypanophobia (fear of medical procedures that involve needles) in individuals, including adults and particularly children.

Unless otherwise defined herein or below in the remainder of the specification, all technical and scientific terms used herein have meanings commonly understood by those of ordinary skill in the art to which the present invention belongs. It is also to be understood that the terminology used herein is for the purpose of describing particular embodiments only, and is not intended to be limiting. In describing and claiming the present invention, the following terminology will be used in accordance with the definitions set out below.

The term “about,” as used herein, indicates the value of a given quantity and can include quantities ranging within 10% of the stated value, or optionally within 5% of the value, or in some embodiments within 1% of the value.

In various embodiments of the present disclosure, the microneedle patch and methods may include vaccine compositions comprising one or more antigens. Non-limiting examples of vaccine compositions include those for influenza, COVID-19, measles, diphtheria, tetanus, pertussis, dengue, hepatitis A, hepatitis B, mumps, human papillomavirus, pneumococcal, meningococcal, rotavirus, polio, smallpox, monkeypox, whooping cough, tuberculosis, meningitis, yellow fever, varicella, rubella, RSV, zoster/shingles, Hib, dengue, typhoid, anthrax, cholera, Japanese encephalitis, rabies, tuberculosis, and/or yellow fever antigens. Non-limiting examples of such vaccine compositions and their methods of manufacture thereof are disclosed in U.S. Pat. No. 10,736,840.

In a preferred embodiment, the vaccine composition is in the form of an array of dissolvable microneedles or a coating on a microneedle formed of another (different) material. The vaccine composition in such embodiments becomes solubilized in vivo following insertion of the microneedle into a biological tissue, e.g., into the skin of the individual (e.g., child or infant).

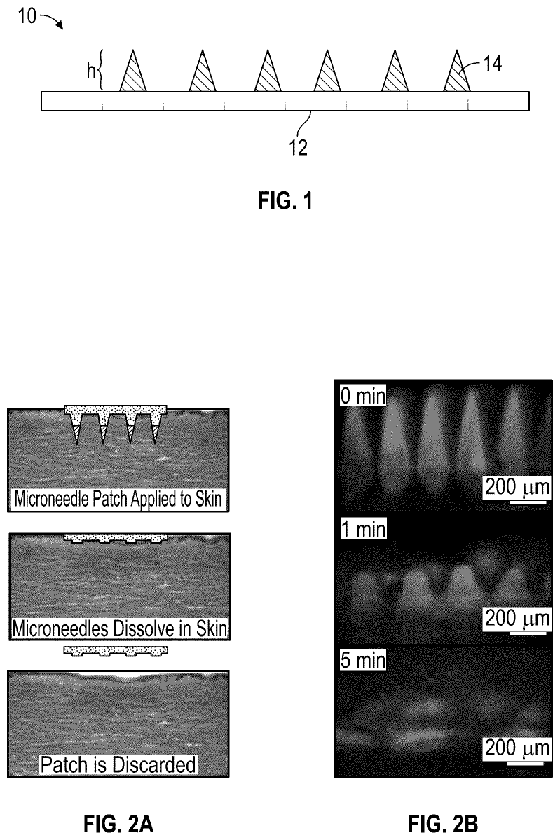

One example of a microneedle array with a plurality of dissolvable microneedles is illustrated in FIG. 1. The microneedle array 10 includes a base substrate 12 with a plurality of microneedles 14. In embodiments, the plurality of microneedles have a height from about 100 μm to about 2000 μm, from about 100 μm to about 1500 μm, from about 100 μm to about 1000 μm, or from about 500 μm to about 1000 μm. The array of microneedles may have any suitable density. For example, the microneedles in the array may be arranged in even or staggered rows, wherein each microneedle is separated from its nearest neighboring microneedle by a distance about equal to the height of the microneedle. The array can include essentially any suitable number of microneedles. In one embodiment, the total mass of vaccine composition in the microneedles of an array is suitable for delivering a prophylactically effective amount of the antigen to a patient. In non-limiting examples, the array may include from 5 to 10,000 microneedles, such as from 50 to 1000 microneedles or from 50 to 200 microneedles.

In some embodiments, the dissolvable microneedles may be formed by casting the vaccine composition in a suitable mold. Various examples of microneedle arrays and their methods of manufacture that may be used to perform the present methods are disclosed in U.S. Pat. Nos. 10,265,511, 10,828,478, 10,940,301, and PCT Application No. PCT/US2023/032664, which are incorporated by reference herein.

In other embodiments, the vaccine composition may be coated onto one or more microneedles comprising a biocompatible material, such as a metal, polymer, or silicone.

In some embodiments, the microneedle patch is applied with the aid of a force feedback indicators (FFI). The FFI may provide audible, tactile, and/or visual feedback indicating that the patch has been properly applied to the skin. For example, the FFI may produce a “snap” which can be heard and/or felt by the user pressing the patch against the skin, and/or the FFI may include a visually discernible change in position of a button or other feature of the patch to indicate whether the patch has been pressed against the skin with sufficient force, for example to lock it into a recessed position as compared to a proud, or elevated, position on the patch. Non-limiting examples of such FFIs are disclosed in U.S. Pat. No. 10,265,511 and PCT Application No. PCT/US2023/032664, which are incorporated by reference herein.

In some other embodiments, the microneedle patch can be applied with the aid of an applicator. The external applicator for a microneedle patch is a device designed to facilitate the application of microneedle patches to the skin. The applicator can contain a base with a skin-side end and a holder for securing the microneedle patch. It may include mechanisms such as spring-loaded systems or other force-generating components to ensure consistent and effective insertion of the microneedles into the skin. The applicator may also feature safety mechanisms to prevent accidental contact with the microneedles and ensure precise delivery of the patch.

The vaccine composition may contain a biologically effective amount of one or more antigens. As used herein, “biologically effective amount” refers to the amount of the one or more antigens needed to stimulate or initiate the desired immunologic response. Thus, the amount of one or more antigens needed to achieve the desired immunological response will necessarily vary depending on a variety of factors including but not limited to the type of antigen, the site of delivery, and the dissolution and release kinetics for delivery of the antigen. In a preferred embodiment, the microneedle patch delivers the antigen in the skin (intradermal). Based on their formulation, the microneedles may dissolve quickly (rapid-release microneedles) and release the vaccine as they dissolve (e.g., the release may start before the microneedles are fully separated from their base) or the microneedles may dissolve slowly or biodegrade (sustained-release microneedles) over hours, days, or weeks to release the antigen over an extended period (which has been shown in some cases to enhance the immune response).

The patch application time (how long the patch is applied to the skin) may be different from the period of antigen-release, for example because in some embodiments, the microneedles are configured to separate from their base. The patch application time may be dictated by how long it takes for the microneedles to separate from their base. In some embodiments, separation occurs from substantially simultaneously with microneedle insertion to minutes, hours or days. In some preferred embodiments, separation occurs from substantially simultaneously with microneedle insertion to about 20 minutes, more preferentially from substantially simultaneously with microneedle insertion to about 5 minutes, and even more preferentially from substantially simultaneously with microneedle insertion to about 1 minute or less.

In some embodiments it is desirable that the vaccine composition be formulated to dissolve in vivo over a period of dissolution from about 1 minute or less to about 60 minutes (FIG. 2A). For example, as shown in FIG. 2B, the microneedles may be substantially dissolved after only 1 minute, and almost fully dissolved after 5 minutes. As used herein, “period of dissolution” or “dissolution period” means the time it takes for the microneedle to be sufficiently wetted during administration such that the microneedle is substantially detached from the base substrate, or in the case of a coating on microneedles, the time it takes for the coating on the microneedle to be substantially detached from the microneedle during administration. In some embodiments, other modes of separation of the dissolvable microneedles may be used, alone or in combination with separation induced by wetting of the microneedle structure or portion thereof. Examples include separation by fracture (which may include application of a shear force), separation at an interface of different materials (see, e.g., U.S. Pat. No. 10,940,301, which is incorporated herein by reference), separation by melting (phase change), or separation including dissolution augmented by inclusion of an effervescent material in a water soluble matrix material forming the microneedle structure or a portion thereof.

In some embodiments, at least 80% of the microneedle dissolves within the period of dissolution period. In some preferred embodiments, more than 90% of the microneedle dissolves within the period of dissolution.

In some embodiments, the antigen is a measles antigen. Although the antigen may take the form of a live virus or an inactivated virus, the measles antigen is typically in the form of a live attenuated virus. In some embodiments, the measles antigen may be combined with a mumps antigen, a rubella antigen, varicella antigen, or combinations thereof (each of which is most commonly in the form of a live attenuated virus).

The amount of the antigen in the vaccine composition may be adjusted to obtain a desired immunologic response. For example, the antigen content in vaccines can be set at at least to 1,000 TCID₅₀ to about 10,000 TCID₅₀ (where TCID₅₀ is defined as median tissue culture infective dose) for a single human dose. For example, a biologically effective amount of vaccine in the composition may be at least 1,000 TCID₅₀.

The vaccine composition provided herein advantageously may be characterized as being stable. As used herein, “stability” of a vaccine composition may be determined by using a tissue culture infective assay (TCID₅₀) after storage for a given time, temperature, and humidity. In some embodiments, the stability of the composition may be shown by the relative activity of the antigen after storage at room temperature or at an elevated temperature of up to 40° C. or greater, as compared to the initial activity of the antigen.

In preferred embodiments, the vaccine formulation exhibits enhanced thermostability, such that the drug delivery device comprising the array of microneedles comprising the antigen can be transported and stored outside the cold chain, or in the cold chain but removed from the cold chain for hours up to days, weeks, months or longer before the device is used in the vaccination methods disclosed herein. This advantageously can facilitate vaccination in remote villages, rural communities, healthcare deserts, and other areas around the world. This can also lower the cost of transporting and storing vaccine. For example, before the drug delivery device is used to administer a measles or other vaccine to the child or infant, the drug delivery device may have been transported or stored outside of cold chain conditions for more than 12 hours and for up to days, weeks, months, or longer. For example, the formulation may remain stable at controlled temperature chain (CTC) conditions of 40° C./75% RH (Relative Humidity) for 14 days, and thus, the formulation may remain stable when stored at temperatures of up to 40° C. during a vaccination campaign or routine immunization, after having been at 5° C. for three months. The formulation may also remain stable so that the vaccine microneedle patch meets various vaccine vial monitor (VVM) requirements such as VVM 30 (i.e., would allow for storage at elevated temperatures of 37° C. for up to 30 days, 25° C. for up to 193 days, and 2-8° C. for >2 years). The formulation may also remain stable after being subjected to freezing (e.g., −20° C.) and thawing (e.g., 25° C.) thus it may withstand scenarios where a vaccine microneedle patch may inadvertently be stored frozen instead of refrigerated or a scenario where it may be stored or left outside the cold chain inadvertently or a scenario in which the temperature inside a refrigerator increases following a power outage or a failure from the refrigerator. The formulation may also remain stable while stored frozen for weeks, months, and years.

In some embodiments, the vaccine formulation is thermostable in an array of microneedles for at least 12 months at a temperature of 5° C. In some embodiments, the vaccine formulation is thermostable in an array of microneedles for at least 12 months at a temperature of 25° C. In some embodiments, the vaccine formulation is thermostable in an array of microneedles for at least 24 months at a temperature of 5° C. In some embodiments, the vaccine formulation is thermostable in an array of microneedles for at least 24 months at a temperature of 25° C. In some embodiments, the vaccine formulation is thermostable for up to 1 month or longer at 37° C. In some embodiments, the vaccine formulation is thermostable for at least 2 weeks under Controlled Temperature Conditions (CTC) of 40° C. or longer. In various embodiments, the vaccine formulation may be thermostable at temperatures from 5° C. to 40° C. or higher, for periods ranging from 1 week to 12 months or more. Refrigerated conditions generally are 5° C.±3° C., so, 5° C. as recited herein may include temperatures from 2° C. to 8° C.

In some particular embodiments, the vaccine formulation is thermostable in an array of microneedles at 25° C. and 65% relative humidity (RH) and/or 37° C. and 75% RH. In some other particular embodiments, the vaccine formulation is thermostable at 25° C. and 60% RH, and/or 40° C. and 75% RH.

In some embodiments, the vaccine formulation is thermostable in an array of microneedles after being subject to freeze-thaw cycles. In one embodiment, thermostability of the vaccine formulation is present after at least three freeze-thaw cycles or as many as at least nine or more freeze-thaw cycles. Thermostability is critical for maintaining adequate vaccine content during the supply chain logistics to deliver and administer vaccinations.

In some embodiments, the vaccination methods described herein may be used to deliver a fractional dose of the vaccine antigen, for example, to deploy a vaccine more quickly, and/or to a larger patient population t than may otherwise be possible in certain situations (e.g., pandemic, limited resources for vaccine production or distribution, etc.).

Administration of a vaccine using a microneedle patch may also be effective to mitigate or overcome trypanophobia, particularly in (but not limited to) infants and toddlers. For example, infants and toddlers are much more likely to tolerate vaccination via a microneedle patch as compared to traditional subcutaneous injections. In various embodiments, administration of vaccines with microneedle patches as disclosed herein may increase the acceptability of vaccinations on an individual basis and/or on a community basis, according to a variety of metrics (as compared to conventional subcutaneous or intramuscular injections, for example) including, but not limited to, decreasing crying by the child or infant receiving the vaccination, decrease or elimination of pain experienced by the patient, perception by parents of decreased or no pain in their child (ren) during administration of the vaccine, decreasing adverse side effects experienced by the patient following administration of the vaccine, increasing the acceptability of non-medical personnel to administer the vaccine, and/or a perception to keep the child or infant healthy. The acceptability survey information described herein may support these metrics.

The microneedle patches disclosed herein may include a single infectious disease antigen, or the microneedles patches may have two or more different infectious disease antigen. The infectious disease antigen can be embedded within a singular excipient formulation. The microneedles can be formulated with the same or different excipient formulations to obtain the desired stability and/or release kinetics. In some embodiments, the microneedle patch can contain microneedles that are made of different matrix materials arranged within the same microneedle. For example, the tip of the microneedle may be formed of a water-soluble matrix containing the infectious disease antigen, and the proximal portion of the microneedle may be formed of a biocompatible polymer or material (different from the water-soluble matrix). Alternatively, the tip of the microneedle may be formed of a water-soluble matrix containing the infectious disease antigen, and the proximal portion of the microneedle may also be formed of a water-soluble matrix, for example, poly(vinyl alcohol) (PVA) and/or sucrose.

The tip of the microneedle is the most distal part (see FIGS. 1-2). In a typical example, approximately two-thirds of the tip portion of the microneedle containing the infectious disease antigen will dissolve, i.e., the “dissolving” portion of the microneedle. The “dissolving” portion of the microneedle can be formulated with water soluble excipients.

Dissolvable microneedles may be formulated with water-soluble excipients, including any such excipients known in the art. For example, the water-soluble excipient may include a sugar or a sugar alcohol, a water-soluble polymer, or a combination thereof. The water-soluble material may include, but is not limited to, dextran, natural polysaccharides, hyaluronic acid, chitosan, beta-sodium glycerophosphate, hydroxypropyl beta cyclodextrin and/or water-soluble polymers, such as poly(vinyl alcohol) (PVA), polyvinyl pyrrolidone (PVP). The water-soluble matrix may also include additional excipients, including but not limited to (i) dextrose, maltose, sorbitol, glycerol, glucose, xylitol, or a combination thereof, (ii) glycerol and/or (iii) a buffer, such as HEPES, phosphate buffer, Tris/HCL, potassium phosphate, ammonium acetate, or a combination thereof. In a particular embodiment, the infectious disease antigen can be incorporated into a microneedle formulation that contains gelatin, sorbitol and buffers. For example, the microneedle formulation can include about 15-50% gelatin, such as 15-25%, or more particularly about 20-25% gelatin, for example, about 22% gelatin; about 5-45% sorbitol, such as about 35-45%, or more particularly about 40-45% sorbitol, for example, about 22% sorbitol. The gelatin:sorbitol ratios can range from 1:2 to 4:1. For example, the gelatin:sorbitol ratio can be 1:2, 2:1, or 4:1 in the microneedle formulations. Sucrose can also be included, for example, about 1-10% sucrose, such as about 1-5% sucrose and optionally other excipients, buffers and/or stabilizers can also be included.

In a non-limiting, particular embodiment, the measles and rubella antigens can be formulated in a microneedle that includes the components listed in the table below:

| | Amount in drug product | | | Quality |
|---|---|---|---|---|
| Component | (microneedles) | | Function | standard |
| Measles | 3.0 | Log10TCIDso | Drug substance | GMP bulk from |
| Rubella | 3.0 | Log10TCIDso | | Serum Institute |
| Excipients | 2.6 | mg | | of India |
| (including gelatin, | | | | |
| sorbitol, buffers, | | | | |
| etc.) | | | | |
| Polyvinyl alcohol | 180 | μg | Bulking, binding, | USP* |
| | | | dissolution agent | |
| Sucrose | 180 | μg | Stabilizer, bulking, | NF* |
| | | | binding, dissolution | |
| | | | agent | |
| Potassium phosphate, | 1 | μg | Buffer | USP* |
| di basic | | | | |
| Potassium phosphate, | 5 | μg | Buffer | USP* |
| monobasic | | | | |

*Current edition

Embodiments of the present invention may be further understood with reference to the following non-limiting examples.

### EXAMPLES

Microneedle patches (MNP) have been ranked as the highest global priority innovation for overcoming immunization barriers in low- and middle-income countries. Provided in the following Examples is data on the tolerability, safety and immunogenicity of a measles and rubella vaccine (MRV) MNP (MRV-MNP) in adults and children, specifically toddlers and infants.

In adults, toddlers, and infants the MRV-MNP was well tolerated and safe. The immunogenicity of the MRV when administered by MNP was similar to the immunogenicity of the vaccine when administered by needle and syringe.

### Example 1. Study Design, Procedures, and Statistical Analysis

In a single-center, double-blind, double-dummy, randomized, active-controlled study, 120 15-18 month old toddlers, 120 9-10 month old infants, and 45 18-40 year old adults were randomized to receive either a MRV-MNP and a placebo (0.9% sodium chloride) subcutaneous (SC) injection or a placebo MNP and MRV by subcutaneous injection (MRV-SC). Solicited local and systemic adverse events were collected for 14 days. Unsolicited adverse events were collected to day 180. Serum neutralizing antibodies to measles and rubella and measles and rubella serum IgG binding antibodies were measured at baseline, day 42 and day 180.

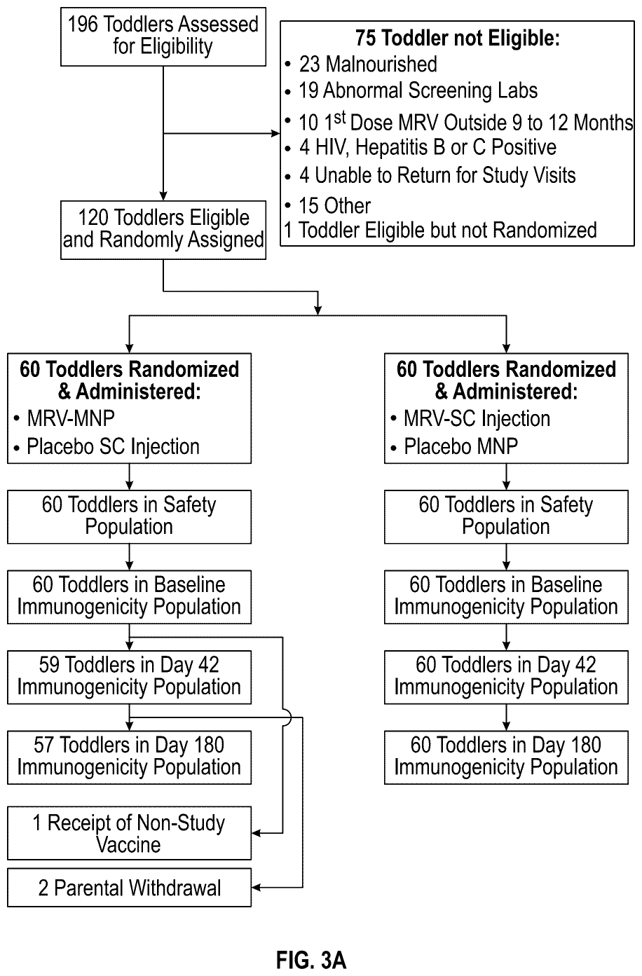

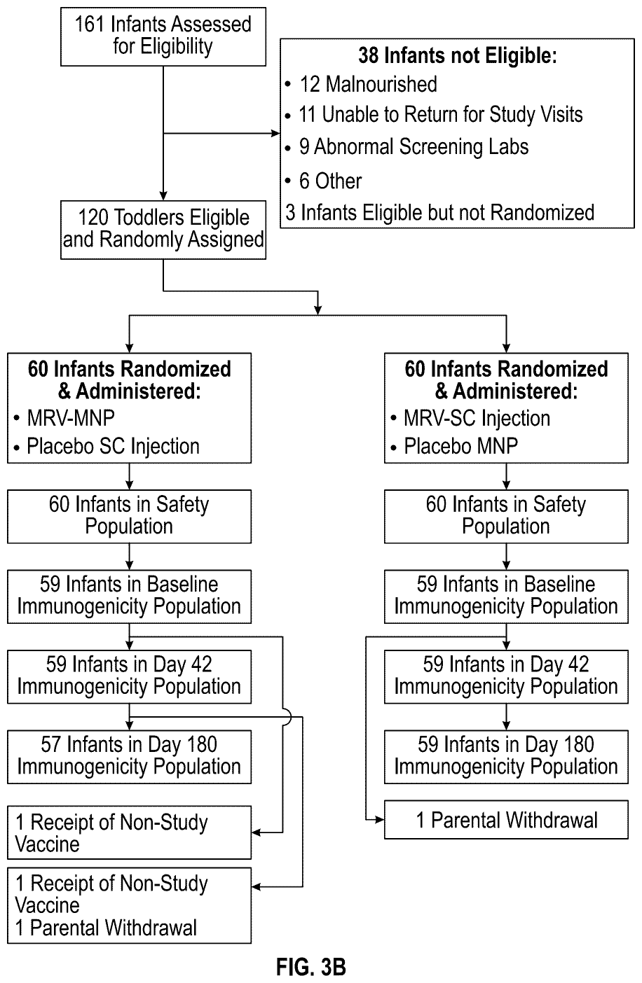

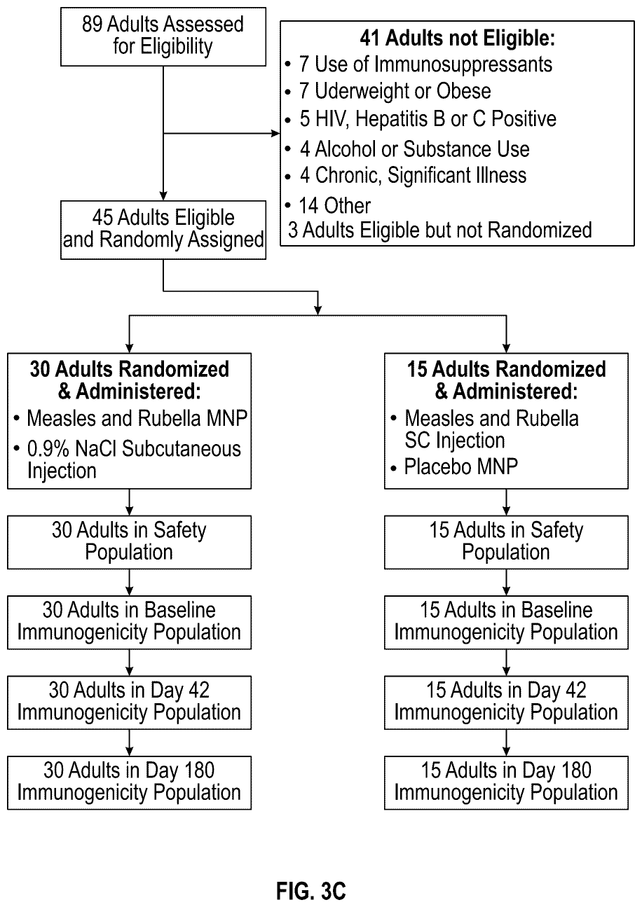

Cohorts of 196 toddlers, 161 infants, and 89 adults were screened in series for enrolment in the study. Of the toddlers, 120 (61.2%) were randomized and vaccinated (FIG. 3A). Fifty-nine of 60 toddlers (98.3%) who received the MRV-MNP and all toddlers (60/60; 100.0%) who received the placebo MNP were included in the primary immunogenicity population. Of the infants, 120 (74.5%) were randomized and vaccinated (FIG. 3B). Fifty-nine of 60 infants (98.3%) in each of the groups were included in the primary immunogenicity population. Of the adults, 45 (50.1%) were randomized and vaccinated (FIG. 3C). All adults in each group were included in the primary immunogenicity population. All vaccinated participants were included in the safety population.

At visit 0, participants were screened for eligibility and blood samples were collected for baseline safety bloods and immunogenicity endpoints. At visit 1, within 2 weeks of the screening visit, participants had final eligibility confirmed, were randomized, and had the MNP administered followed by the SC injection. Participants had additional clinic visits on day 7 (visit 2) and day 14 (visit 3) during which solicited adverse event data were reviewed, any new unsolicited adverse events were recorded, and safety bloods (visit 2) were collected. Additional clinic visits took place on day 42 (visit 4) and day 80 (visit 5) during which blood samples were collected for immunogenicity endpoints and unreported adverse events were recorded. Additional immunizations according to the routine schedule were given to participants on or after the day 42 visit.

Both the MRV-MNP (Micron Biomedical, Inc. Atlanta, United States) and the single 0.5 mL dose of the MRV for SC injection (Serum Institute of India Pvt. Ltd., Pune, India) contained not less than 1000 CCID₅₀ of the live-attenuated Edmonston-Zagreb measles virus and not less than 1000 CCID₅₀ of the live-attenuated Wistar RA 27/3 rubella virus. The bulk vaccine viruses (Serum Institute on India Pvt. Ltd) were incorporated into dissolvable microneedles made up of pharmaceutical-grade excipients found in the U.S. FDA's Inactive Ingredient Database for Approved Drug Products. The MRV-MNPs were designed to deliver similar doses of the vaccine to those delivered by the SC injection. The placebo MNPs contained the same excipients as those contained in the MRV-MNP, but without the vaccine. The placebo for SC injection consisted of 0.5 mL of 0.9% (weight/volume) sterile sodium chloride (Hameln Pharmaceuticals Ltd, UK).

The MNP was applied to the dorsal aspect of the wrist for five minutes then removed. Participants were observed closely throughout this time to prevent the MNP from being disturbed. The SC injection was administered into the thigh in both toddlers and infants. All SC injections were given with a 23 G, 25 mm, 0.5 mL auto-disable needle and syringe.

### Example 2. Microneedle Patch Design and Fabrication

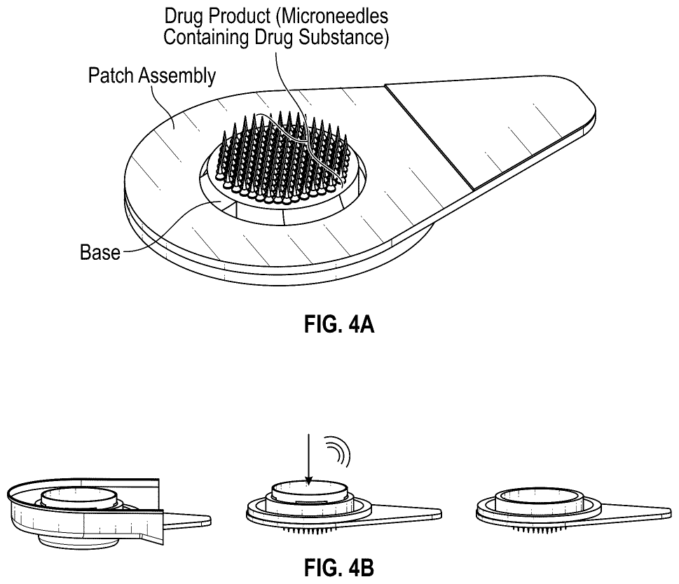

The microneedle patch used to administer the MRV is shown in FIG. 4A. The MRV-MNP is intended to deliver at least 1,000 TCID₅₀ of both the measles and rubella antigens intradermally. The terms “CCID50” and “TCID50” are used interchangeably herein.

The microneedle patch included a base substrate from which a plurality of microneedles extended perpendicularly. The base substrate was attached to a patch assembly that included a force feedback indicator. The microneedles and force feedback indicator was attached to a backing layer via an opening therein. That is, the backing layer included an opening sized and shaped to receive the plurality of microneedles and the FFI within the opening. The base substrate holding the plurality of microneedles was attached to the FFI with a first adhesive layer, and the FFI was attached to the backing layer by a second adhesive layer. The backing layer also included a tab portion which extended laterally away from the microneedles. The tab portion enabled the user to handle the patch without contacting the microneedles.

The MRV-MNP included an array of 163 dissolvable microneedles each having a height of 700 μm, and the microneedles consisted primarily of the measles and rubella antigens and appropriate bulking agents. The microneedle formulation was GMP bulk manufactured by Serum Institutes of India (SSI). The measles and rubella substances were both concentrated and combined. To form the microneedles, the aqueous formulation was deposited into a first portion of a microneedle mold, which is reciprocal to the desired dimensions of the microneedle array. The mold and formulation were then subjected to drying to solidify the formulation and form the array of microneedles.

TABLE 4
Composition of drug product (Microneedles)

| | Amount in drug product | | | Quality |
|---|---|---|---|---|
| Component | (microneedles) | | Function | standard |
| Measles | 3.0 | Log10TCIDso | Drug substance | GMP bulk from |
| Rubella | 3.0 | Log10TCIDso | | Serum Institute |
| Excipients | 2.6 | mg | | of India** |
| Polyvinyl alcohol | 180 | μg | Bulking, binding, | USP* |
| | | | dissolution agent | |
| Sucrose | 180 | μg | Stabilizer, bulking, | NF* |
| | | | binding, dissolution | |
| | | | agent | |
| Potassium phosphate, | 1 | μg | Buffer | USP* |
| di basic | | | | |
| Potassium phosphate, | 5 | μg | Buffer | USP* |
| monobasic | | | | |

*Current edition
**See: https://cdsco.gov.in/opencms/resources/UploadCDSCOWeb/2018/UploadSmPC/77.%20MR%20vaccine%20Serum.pdf and SPC-II-lab-pl Template

Next the base substrate, from which the microneedles extend, was formed. It did not include the measles or rubella antigens and was composed only of appropriate bulking agents. After the microneedles were dried, the base formulation was deposited into a second portion of the mold, which was reciprocal to the desired dimensions of the base substrate. The mold was then dried to solidify both the microneedle and base formulations, such that the microneedles were integrally formed with the base substrate.

The composition of the drug product for the measles-rubella vaccine microneedle patch (MRV-MNP) used in the Phase 1/2 trial is set forth in the table below. The drug product is defined as the portion of the microneedles that is designed to penetrate and dissolve in the skin.

The FFI was then attached to the base with hypoallergenic tape and to an adhesive backing, formed of a hypoallergenic foam medical tape. The FFI included three layers: two plastic (high impact polystyrene, HIPS) components and a stainless steel dome. The FFI provided tactile and audible feedback from the buckling of the stainless steel dome when the MRV-MNP was applied with the requisite minimum application force. The FFI also provided visual feedback, irreversibly hiding the colored sidewalls of the FFI cover/button of the FFI after application of an effective insertion force.

As shown in FIG. 4B, the MRV-MNP was packaged with a protective cap covering the microneedle array prior to use (left). During use, the MRV-MNP was removed from the cap and applied to the skin (middle), and sufficient force was applied to enable microneedle insertion into the skin. FIG. 4B shows the MRV-MNP with a FFI included, producing an audible click when enough force is applied, locking it in a depressed position within a housing hiding the colored sidewall, serving as visual indications that the MRV-MNP has been used. The MRV-MNP patch was then worn on the skin for 5 minutes to dissolve the microneedles into the skin to deliver the vaccine intradermally.

Table 5 provides an illustrative example of relevant product attributes of the MRV-MNP product.

TABLE 5
MRV-MNP Product Attributes.

| Key Product Attributes | Specifications |
|---|---|
| Presentation | Single dose affixed to medical-grade blister |
| | cap |
| Dose | 10⁴ TCID₅₀ for measles and rubella |
| Storage | 2° C. to 8° C. |
| Stability | 2 years at 2° C. to 8° C. |
| Packaging | Foil pouch with desiccant |
| Wear Time | 5 minutes |

Table 6 provides an illustrative example of relevant characteristics of an MN array and backing.

TABLE 6
MN Array and Backing Characteristics.

| | | |
|---|---|---|
| MN Array | MN Geometry | Conical |

| MN Height | 700 | μm |
|---|---|---|
| Array Shape | Circular | |
| Array Size | ~0.8 | cm² |

| | |
|---|---|
| Backing | Medical adhesive, hypoallergenic |
| | Tear drop shape, ~3.5 cm long |
| | Tab for handling |
| | Force-feedback indicator |

### Example 3. Safety Results and Analysis

Solicited systemic adverse events and local adverse events at the MNP application site and SC injection site were collected and graded for severity on the day of administration (day 0) and for a further 13 days, through home visits conducted by trained field workers. Unsolicited adverse events and serious adverse events were collected from the day of study product administration until day 180, categorized by preferred term according to the Medical Dictionary of Regulatory Affairs and graded for severity.

The safety outcomes were the incidence and severity of solicited local and systemic adverse events on the day of study product administration (day 0) and for a further 13 days; the incidence and severity of unsolicited adverse events including serious adverse events, from the day of study product administration until 180 days after study product administration; the incidence and severity of biochemical and hematological laboratory abnormalities on day 7 and, in adults only, day 14 after study product administration. The relatedness of solicited systemic adverse events, unsolicited adverse events, and laboratory abnormalities, to study product administration was assessed.

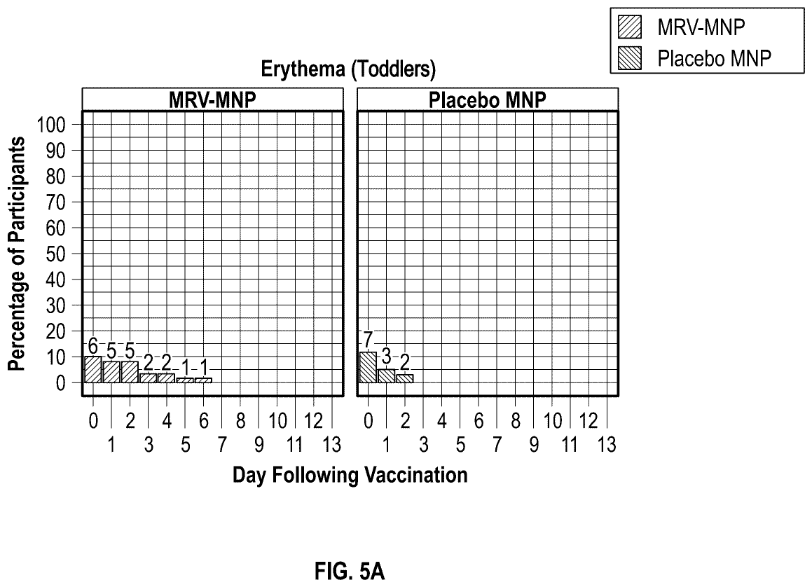

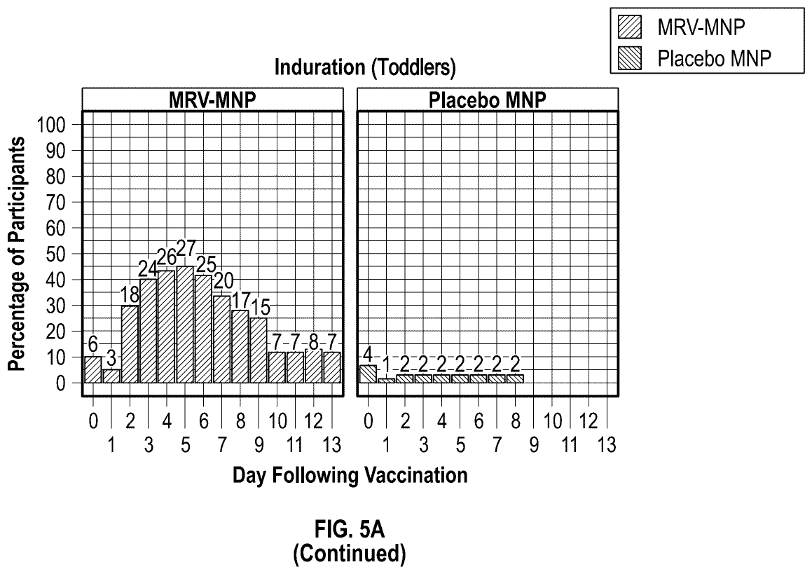

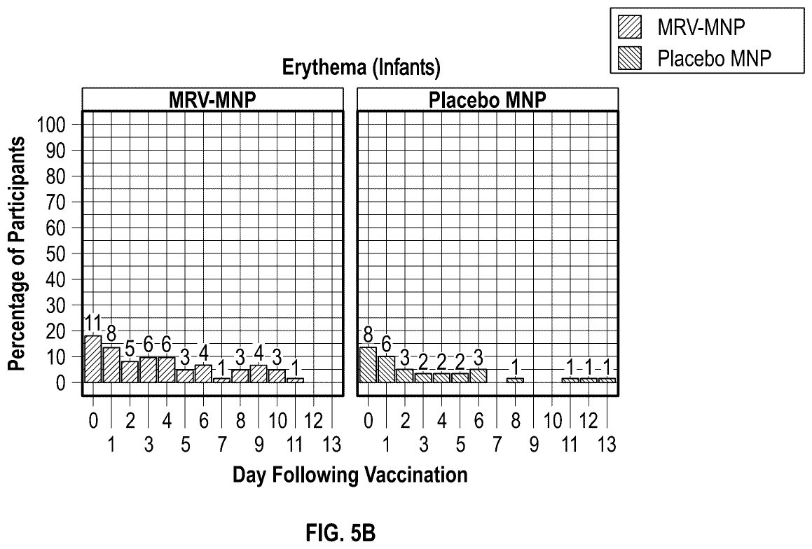

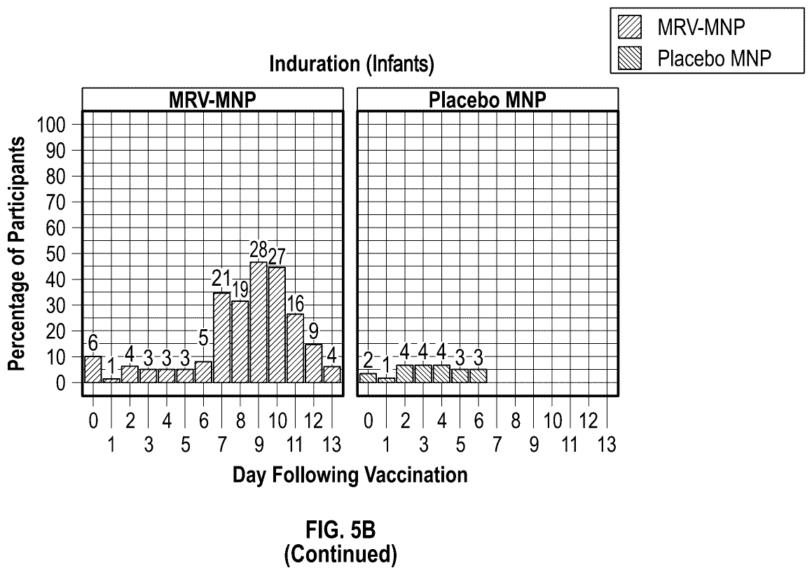

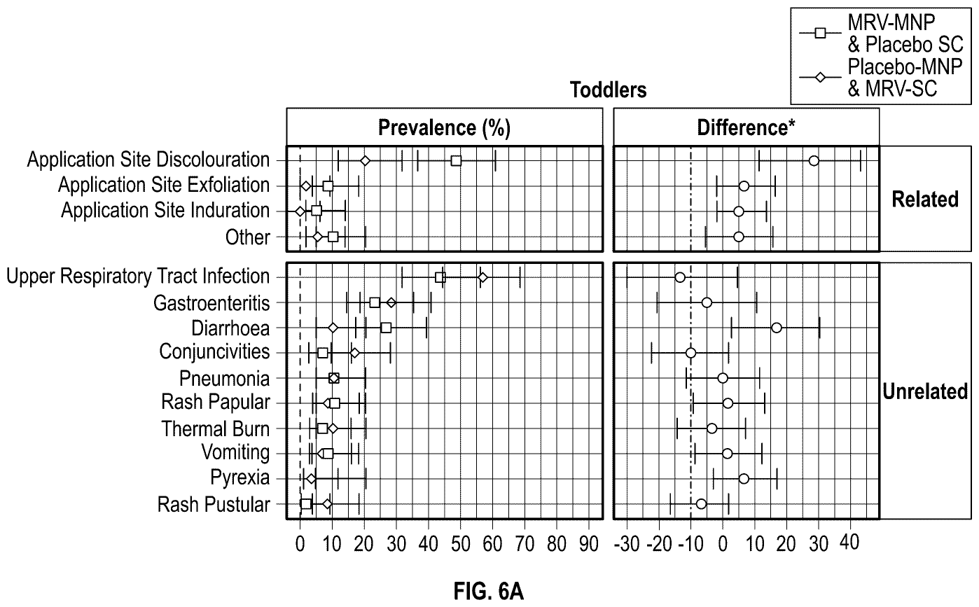

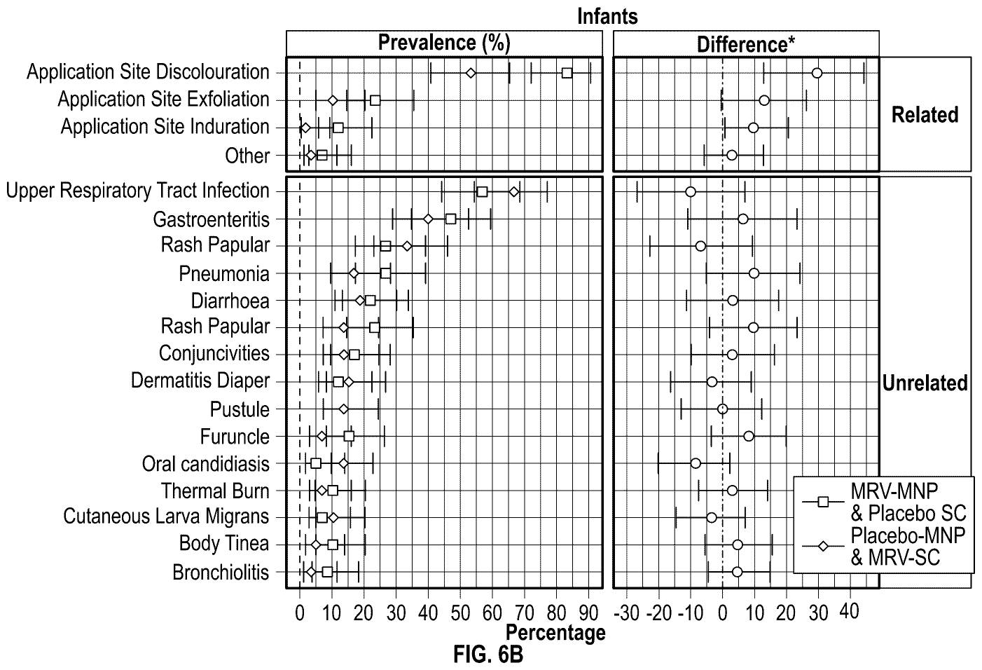

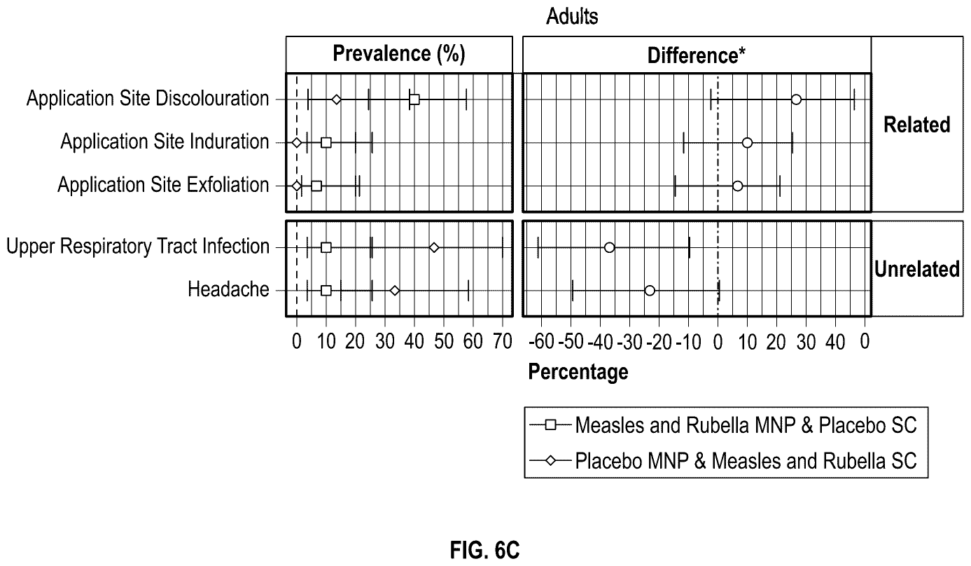

There were no acute allergic reactions in toddlers. Fifty toddlers (83.3%) had a mild local reaction at the MRV-MNP application site compared to 18 (30.0%) at the placebo MNP application site. Mild induration was the most common local reaction and was experienced by 46 (76.7%) of toddlers who received the MRV-MNP compared to 9 (15.0%) of those who received the placebo MNP. The incidence of mild induration in toddlers peaked at 45.0% (27/60) on day five following MRV-MNP application (FIG. 5A). Five toddlers (8.3%) had a fever following MRV-MNP compared to 11 (18.3%) in the MRV-SC group. There was one severe fever (≥39.0° C.) in a toddler in the MRV-SC group. Twenty-seven toddlers (45.0%) in the MRV-MNP group experienced a mild or moderate solicited systemic adverse event compared to 30 (50.0%) in the MRV-SC group. Fifty-nine toddlers (98.3%) experienced at least one of the 203 unsolicited adverse events reported following MRV-MNP compared to 56 toddlers (93.3%) who experienced at least one of the 187 unsolicited adverse events reported in the MRV-SC group. Although more diarrhea was reported in the MRV-MNP group, this is unlikely to be of significance as an isolated finding (FIG. 6A). There was one (1.7%) serious adverse event in toddlers who received the MRV-MNP compared to eight events in seven toddlers (11.7%) in the MRV-SC group. Thirty-five toddlers (58.3%) experienced at least one related adverse event following MRV-MNP administration compared to 16 toddlers (26.7%) following the placebo MNP (FIG. 6A). Discoloration at the patch application site was the most common related adverse event and was reported in 29 toddlers (48.3%) in the MRV-MNP group and 12 (20.0%) in the placebo MNP group (FIG. 6A). All local related events were mild in severity. There was one moderate related adverse event, a generalized papular rash, in the MRV-MNP group. All application site related events in toddlers resolved before day 180. There were no related serious or severe adverse events in the toddler cohort. There were no clinically significant, related changes in laboratory parameters in either group.

There were no acute allergic reactions in infants. Forty-six infants (76.7%) had a mild local reaction at the MRV-MNP application site compared to 18 (30.0%) at the placebo MNP application site. Mild induration at the MNP application site was the most common local reaction and was experienced by 39 infants (65.0%) who received the MRV-MNP compared to 6 (10.0%) of those who received the placebo MNP. The incidence of mild induration in infants peaked at 46.7% (28/60) on day nine following MRV-MNP application (FIG. 5B). There were no moderate or severe reactions at either MNP application site. Eight infants (13.3%) in the MRV-MNP group had a fever compared to 4 (6.7%) in the MRV-SC group. There were no severe fevers in the infant cohort. Thirty-one infants (51.7%) in the MRV-MNP group experienced a mild or moderate systemic adverse event compared to 24 (40.0%) in the MRV-SC group. All infants in the MRV-MNP group experienced at least one of the 347 unsolicited adverse events reported compared to 59 infants (98.3%) who experienced at least one of the 285 unsolicited adverse events in the MRV-SC group. There were no notable trends in the incidence of specific adverse events, judged to be unrelated to vaccination, comparing the two groups (FIG. 6B). There was one (1.7%) serious adverse event in an infant in each group. Fifty-seven infants (95.0%) experienced at least one related adverse event in the MRV-MNP group compared to 38 infants (63.3%) following the placebo MNP (FIG. 6B). Discoloration at the MNP application site (all hyperpigmentation) was the most common related adverse event and was reported in 50 infants (83.3%) in the MRV-MNP group and 32 (53.3%) in the placebo MNP group on day 14. Five infants (8.3%) in the MRV-MNP group had ongoing application site discoloration on day 180. All related events in the placebo MNP group had resolved at the same time point. There were no related serious or severe adverse events in the infant cohort. There were no clinically significant, related changes in laboratory safety parameters in the infant, toddler or adult groups. No notable changes from baseline were observed for any parameter in all cohorts.

Table 7 below provides an illustrative example of the safety laboratory results from the infant cohort.

TABLE 7
Laboratory Results on Safety in the Infant Cohort.
Mean change in Day 7 post vaccination safety laboratory
results from baseline - infant cohort

| | MRV-MNP, PLA-SC | | MRV-SC, PLA-MNP | |
|---|---|---|---|---|
| | Change | 95% CI | Change | 95% CI |
| Haemoglobin | −0.22 | −0.36; −0.08 | −0.12 | −0.27; 0.03 |
| (g/dL) | | | | |
| WBC | −0.71 | −1.43; 0.01 | −1.32 | −1.87; −0.7 [symbol] |
| (×10 [symbol] /L) | | | | |
| Platelets | −63.5 | −101.0; −26.0 | −37.4 | −78.7; 3.9 |
| (×10 [symbol] /L) | | | | |
| Sodium | 0.6 | 0.1; 1.1 | 1.0 | 0.6; 1.6 |
| (mmol/L) | | | | |
| Potassium | −0.06 | −0.19; 0.07 | −0.17 | −0.28; −0.06 |
| (mmol/L) | | | | |
| Calcium | −0.06 | −0.10; −0.02 | −0.05 | −0.09; −0.04 |
| (mmol/L) | | | | |
| Albumin | −0.2 | −1.2; 0.7 | 0.2 | −0.7; 1.2 |
| (g/L) | | | | |
| Total Protein | −0.9 | −2.2; 0.4 | −1.0 | −2.7; 0. [symbol] |
| (g/L) | | | | |
| Total Bilirubin | −0.54 | −1.14; 0.05 | −0.48 | −0.91; −0.04 |
| (μmol/L) | | | | |
| ALT | −0.9 | −3.2; 1.3 | −1.7 | −4.1; 0.7 |
| (U/L) | | | | |
| AST | 2.1 | −0.4; 4.7 | −0.1 | −2.4; 2.2 |
| (U/L) | | | | |
| Creatinine | 1.4 | 0.6; 2.3 | 1.9 | 1.0; 2.8 |
| (μmol/L) | | | | |
| Urea BUN | 0.03 | −0.08; 0.13 | −0.07 | −0.17; 0.03 |
| (mmol/L) | | | | |

[symbol]

indicates data missing or illegible when filed

There were no acute allergic reactions in adults. Eight adults (26.7%) had a mild local reaction at the MRV-MNP application site compared to three (20.0%) at the placebo MNP application site. Pruritis was the most common local reaction among those who experienced the MRV-MNP and was experienced by five adults (16.7%), where mild pain was the most common reaction of those receiving the placebo and was experienced by two adults (13.3%). There were no moderate or severe reactions at either MNP application site. No adults in either group experienced a fever. Fifteen adults (50%) in the MRV-MNP group experienced a mild or moderate systemic adverse event compared to seven adults (46.7%) in the MRV-SC group. There were no related serious or severe adverse events in the adult cohort.

Induration at the MRV-MNP application site was the most frequent local reaction occurring in 46/60 (76.7%) of toddlers and 39/60 (65.0%) of infants. Related unsolicited adverse events, most commonly discoloration at the application site, were reported in 35/60 (58.3%) of toddlers and 57/60 (95.0%) of infants that had received the MRV-MNP. All local reactions were mild. There were no related severe or serious adverse events.

Given the delivery of the vaccine viruses into the skin, mild local reactions at the MRV-MNP application site were more frequent than at the SC injection site. Induration was the most common local solicited event in toddlers and infants. The earlier peak in incidence in the toddlers (day 5) compared to the infants (day 9) suggests an anamnestic infiltration of cells primed by the previous measles and rubella vaccination in the toddlers, while the timing of the response in the infants is consistent with a naïve response driven by viral replication. Despite this, it is reassuring that the all the reactions were mild and of no safety concern in either group.

Application site hyperpigmentation occurred most frequently following the MRV-MNP, although also occurred following placebo MNP, so was not solely driven by responses to the vaccine viruses. However, diverse inflammatory and other soluble mediators have been shown to increase melanin production, while post-inflammatory hyperpigmentation is also more frequent in dark skin.

### Example 4. Efficacy Results and Analysis

Serum was separated from blood samples, collected at baseline (visit 0), day 42 (visit 4) and day 180 (visit 5), and frozen at below −70° C. within four hours, prior to immunogenicity testing by the U.S. Centers for Disease Control and Prevention laboratories. Measles virus serum neutralizing antibodies (SNA) were measured using a plaque reduction neutralization test based on a WHO-recommended protocol. Rubella virus SNA were measured using a direct immunocolorimetric assay. Measles and rubella virus immunoglobulin G (IgG) was measured using a multiplex bead array. All antibody results were calibrated to appropriate WHO standards.

The immunogenicity outcomes were assessed using both SNA and IgG binding antibodies to measles and rubella and were seroconversion rates (the percentage of participants who were seronegative at baseline and seropositive at day 42); rates of four-fold antibody rise (the percentage of participants who were seropositive at baseline and who had a four-fold increase in antibody concentrations by day 42); immune response rates (combining the number of participants undergoing seroconversion and the number experiencing a four-fold rise in antibodies); the percentage of participants who were seropositive and the geometric mean antibody concentrations (GMC) at day 42 and day 180; the geometric mean fold rise (GMFR) in antibody concentrations between baseline and day 42. Seropositivity was defined as an antibody concentration of ≥200 mIU/mL for measles and ≥10 IU/mL for rubella.

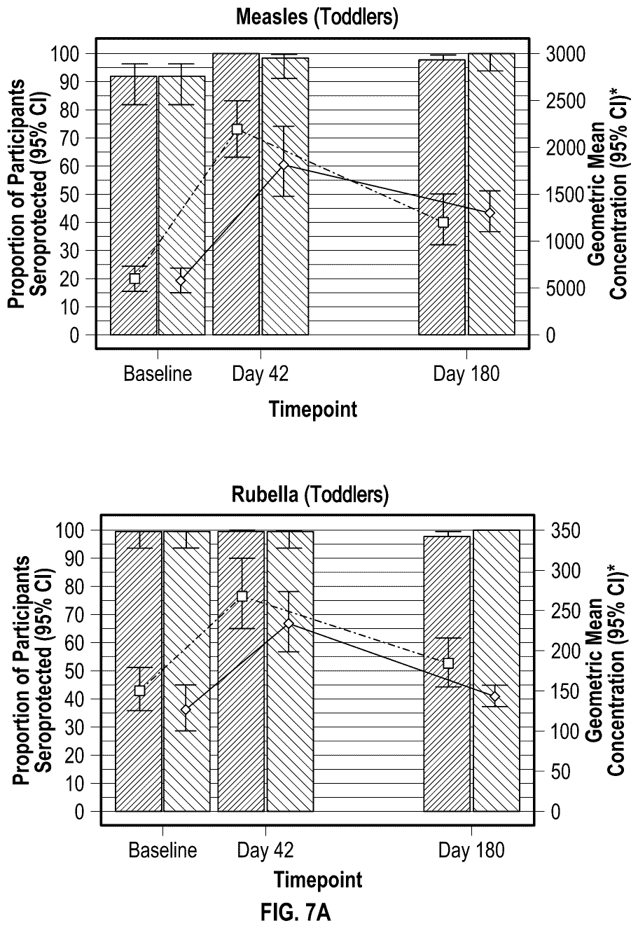

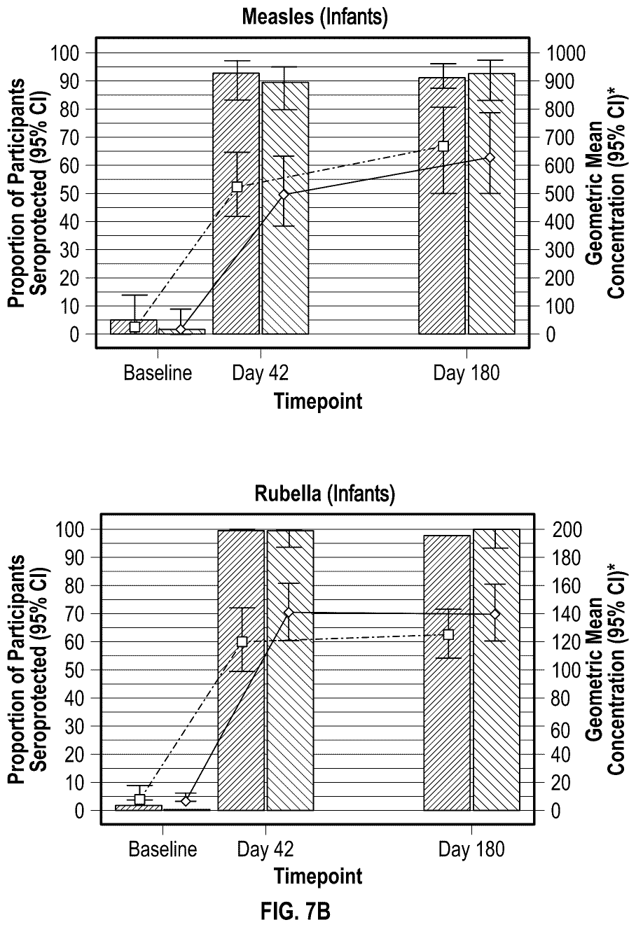

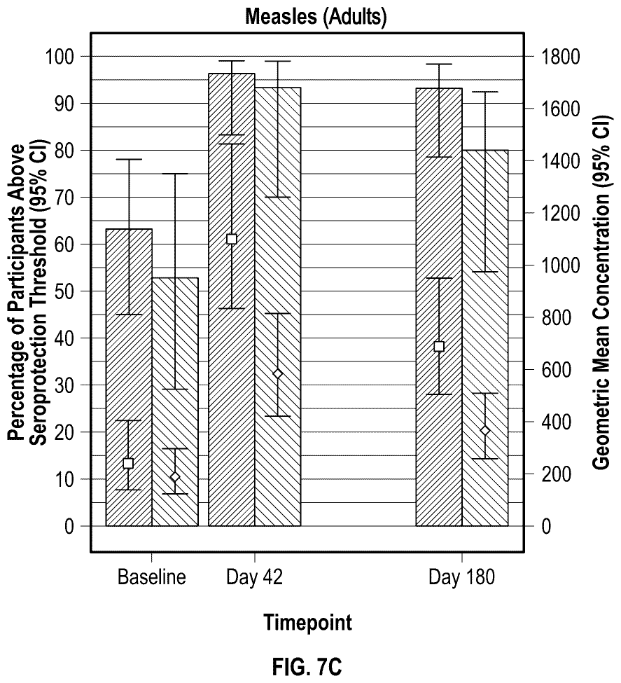

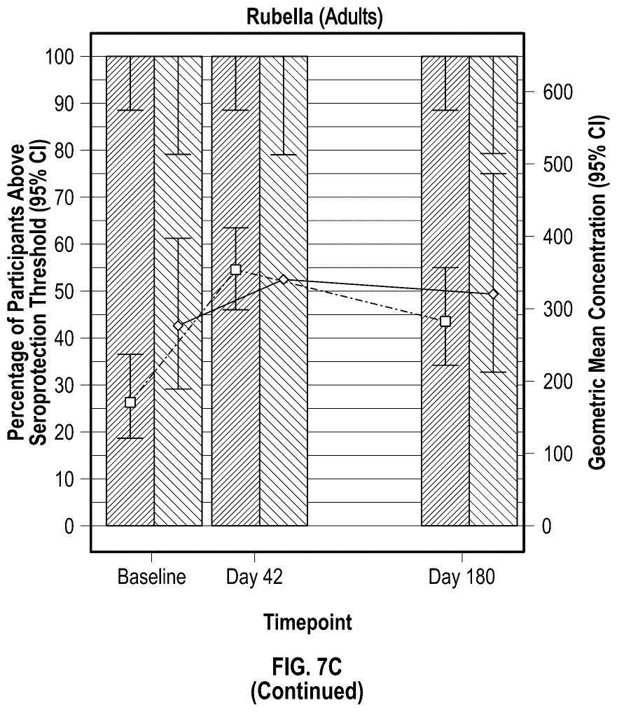

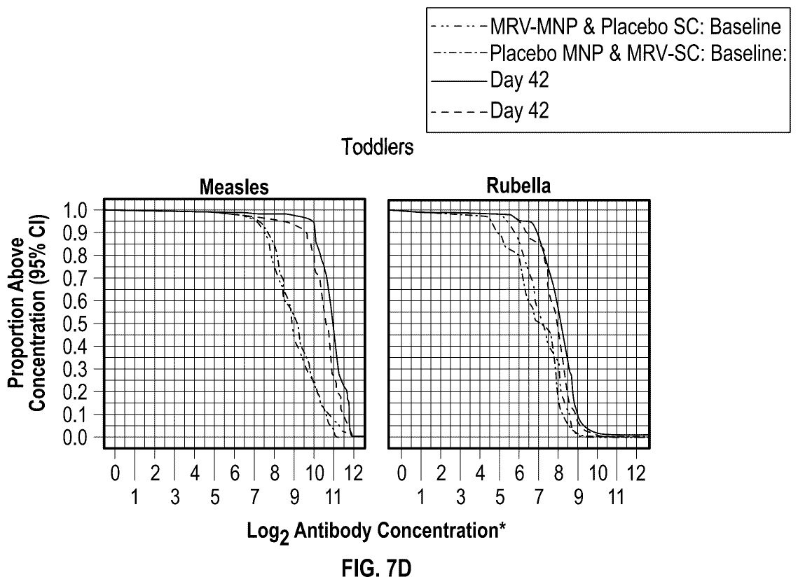

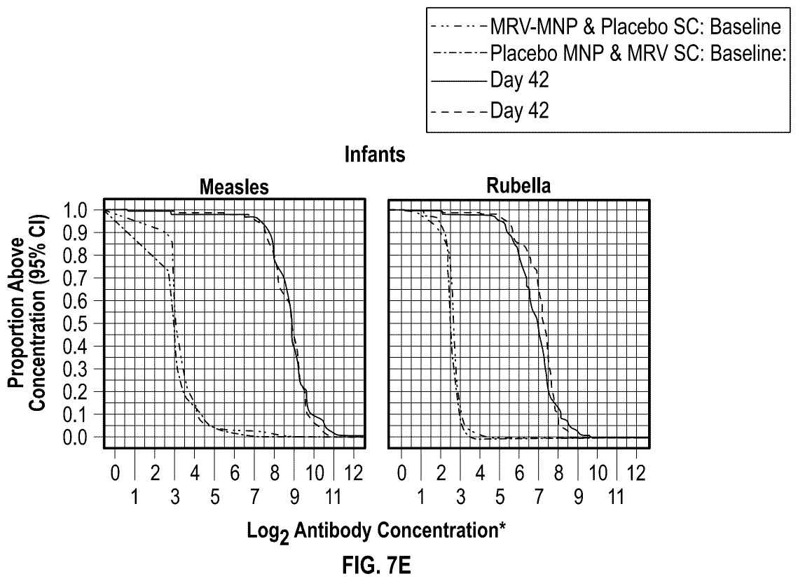

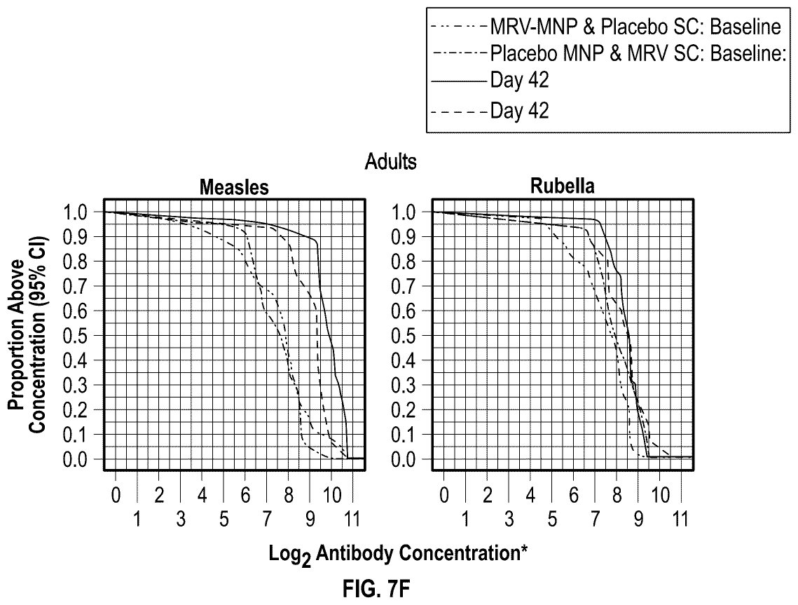

There were no notable differences in measles serological endpoints between groups in toddlers at baseline based on SNA (FIG. 7A). Over 91% of toddlers in both groups were seroprotected. Measles GMC went from 572.8 mIU/mL (95% CI 450.1-729.1) at baseline to 2182.9 mIU/mL (95% CI 1905.6-2500.5) on day 42 (GMFR 3.8 [95% CI 3.0-4.9]) in the MRV-MNP group and from 566.9 mIU/mL (95% CI 448.8-716.1) at baseline to 1811.5 mIU/mL (95% CI 1480.6-2212.4) on day 42 (GMFR 3.2 [95% CI 2.5-4.1]) in the MRV-SC group. In total, 47.5% (28/59; 95% CI 35.27-59.96) of toddlers had an immune response to measles in the MRV-MNP group compared to 41.7% (25/60; 95% CI 30.06-54.27) in the MRV-SC group. Measles GMC remained substantially above baseline concentrations at day 180.

Three of 59 (5.1% [95% CI 1.74-13.92]) infants in the MRV-MNP group and one of 59 (1.7% [95% CI 0.30-9.00]) in the MRV-SC group were seroprotected against measles at baseline (FIG. 7B). Seroconversion occurred in 92.9% (52/56 [95% CI 83.02-97.19]) of infants in the MRV-MNP group compared to 89.7% (52/58 [95% CI 79.21-95.17]) in the MRV-SC group. Measles GMC on day 42 were 520.9 mIU/mL (95% CI 420.8-644.9) in the MRV-MNP group and 495.2 mIU/mL (95% CI 402.5-609.3) in the MRV-SC group. In infants, the SNA GMCs continued to increase to day 180 in both groups. Seroprotection rates at day 180 were 91.2% (52/57 [95% CI 81.06-96.19]) in the MRV-MNP group and 93.2% (55/59 [95% CI 83.82-97.33]) in the MRV-SC group.

All the toddlers were rubella seroprotected at baseline based on SNA concentrations (FIG. 7A). Rubella GMC went from 151.6 IU/mL (95% CI 126.2-182.2) at baseline to 268.2 IU/mL (95% CI 228.3-315.0) on day 42 (GMFR 1.8 [95% CI 1.4-2.2]) in the MRV-MNP group and from 126.4 IU/mL (95% CI 101.2-157.9) at baseline to 234.3 IU/mL (95% CI 199.6-274.9) on day 42 (GMFR 1.9 [95% CI 1.5-2.9]) in the MRV-SC group. Reflecting high baseline antibody titers, less than 14% of toddlers had an immune response to rubella across both groups (FIG. 7D). Rubella GMC returned towards baseline levels in both groups by day 180.

One of 59 (1.7% [95% CI 0.30-9.00]) infants in the MRV-MNP group was seroprotected against rubella (FIG. 7B) at baseline. All infants in the MRV-SC group were seronegative. All infants in both groups seroconverted to rubella by day 42 (MRV-MNP group-58/58 [100.0%; 95% CI 93.79-100.00], MRV-SC group-59/59 [100.0%; 95% CI 93.89-100.00]) and remained seropositive at day 180. Rubella GMCs on day 42 were 120.3 IU/mL (95% CI 99.9-144.9) in the MRV-MNP group and 140.3 IU/mL (95% CI 120.9-162.7) in the MRC-SC group and were similar at day 180. Reverse cumulative distribution curves illustrate the aligned of the antibody distribution between groups before and after vaccine administration (FIG. 7E). In infants, 92.9% (52/56; 95% CI 83.02-97.19) seroconverted to measles and 100.0% (58/58; 95% CI 93.79-100.00) seroconverted to rubella following MRV-MNP administration while 89.7% (52/58; 95% CI 79.21-95.17) and 100.0% (59/59; 95% CI 93.89-100.00) seroconverted to the same viruses following MRV-SC.

The doses of the attenuated viruses delivered by MNP and SC injection were similar. The measles seroconversion rate of over 92% in infants who had the vaccine delivered by MNP is comparable to the rates reported following SC delivery of the vaccine. Between 85% and 90% of infants are expected to seroconvert following vaccination at nine-months, with the figure rising to 90% to 95% in children vaccinated at 12-months-of-age. Similarly, a recent meta-analysis reported a seroconversion rate of 99% (95% CI 98 to 99) in children following a single dose of a rubella vaccine containing the RA 27/3 strain.

### Example 5. Dissolution Results and Analysis

The remaining portions (including the base substrate) of the MRV-MNP administered in the study were returned for analysis following completion of the study. The MRV-MNP were inspected using light microscopy. Each microneedle within each patch was given a percent of microneedle height dissolution score, representing the amount of the microneedle height that was dissolved during administration. All of the microneedle dissolution scores of a given array were averaged to provide an average microneedle height dissolution for each patch used in the study.

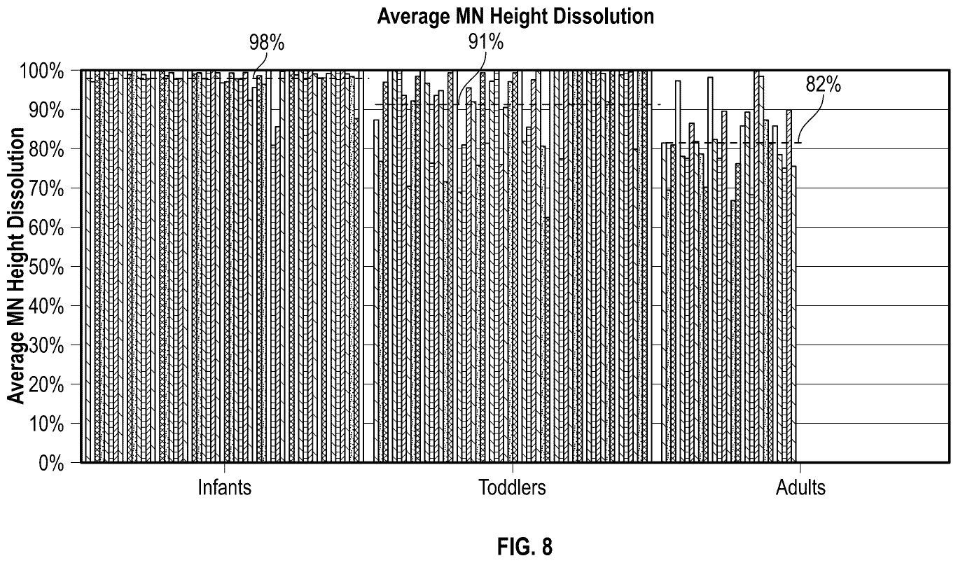

As shown in FIG. 8B, the average microneedle height reduction was 98% in patches administered to the infant cohort, 91% in patches administered to the toddler cohort, and 82% for patches administered to an adult cohort.

### Example 6. Stability Results and Analysis

For stability studies, MRV-MNPs were fabricated and then stored under controlled conditions. They were tested at specific time points according to a stability protocol. At each time point, three MRV-MNPs were tested. For technical and clinical batches, testing consisted of appearance, potency, bioburden and moisture. For the other stability studies (performed after the clinical trial), only potency was measured.

An example of a stability plan for clinical batches is set forth in the following table:

TABLE 8
Stability plan for the MRV-MNP Clinical
Batches to be used in the clinical trial

| | Storage | | | | |
|---|---|---|---|---|---|
| | condition | Storage time (months)* | | | |
| Test | ° C. | 0 | 1 | 3 | 6 |
| Appearance | 5 ± 3 | All tests performed at | T | T | T |
| Potency | | Release in addition to | T | T | T |
| Bioburden | | identity, content uniformity, | — | — | T |
| Moisture | | MN height, & endotoxin | T | T | T |

*Stability at start, middle, and end of clinical study enrollment estimated at 1, 3, and 6 months.
T Testing scheduled according to stability program.
— No testing scheduled according to the stability program.

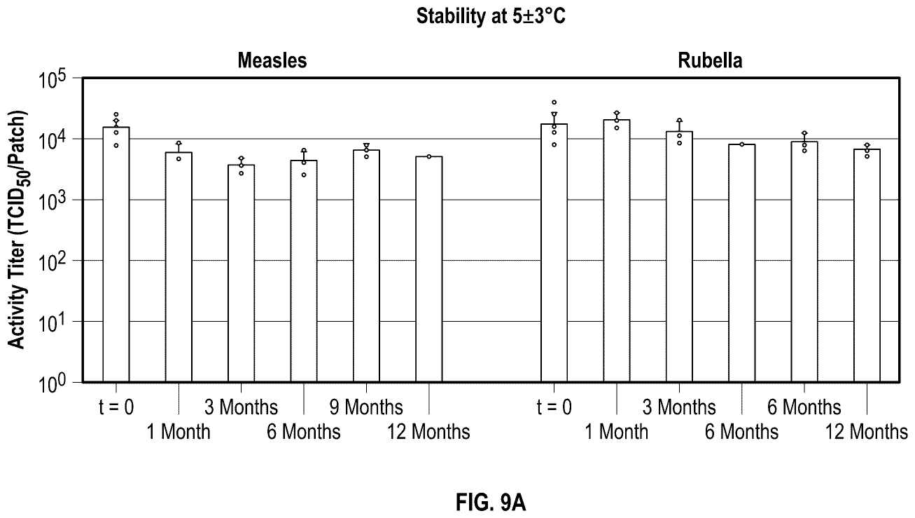

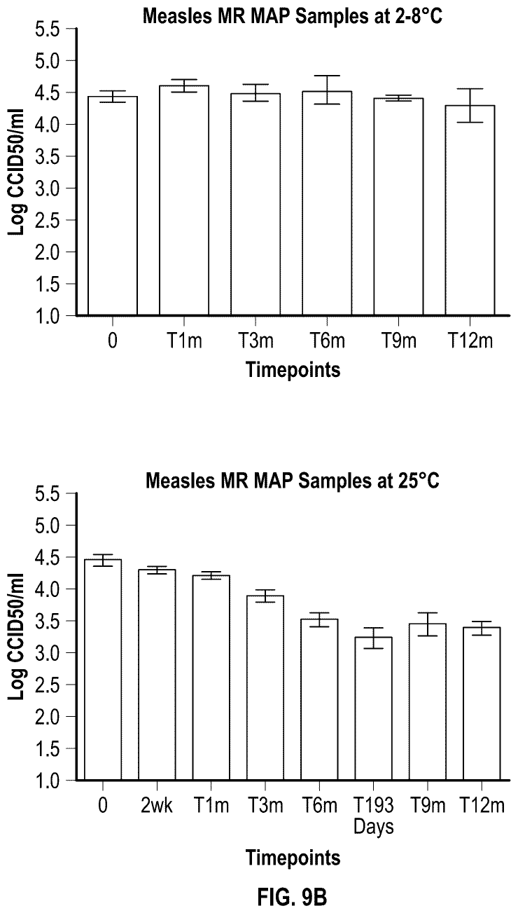

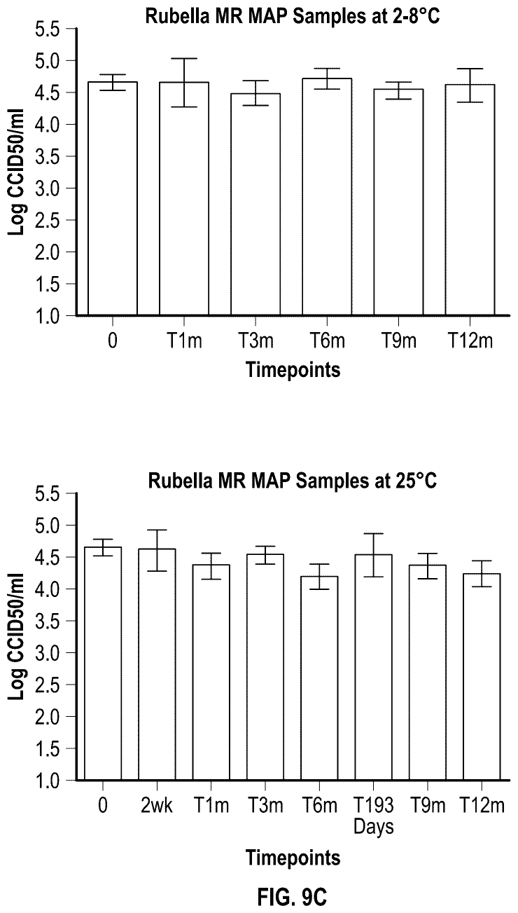

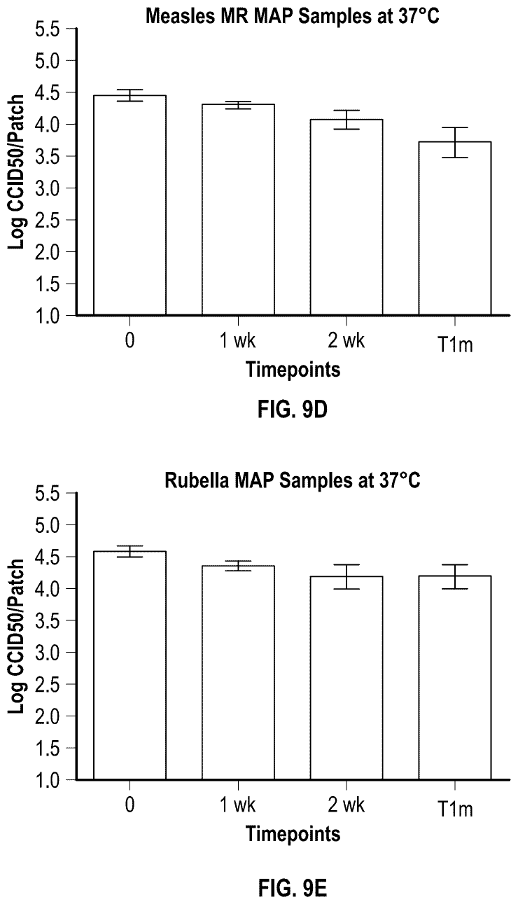

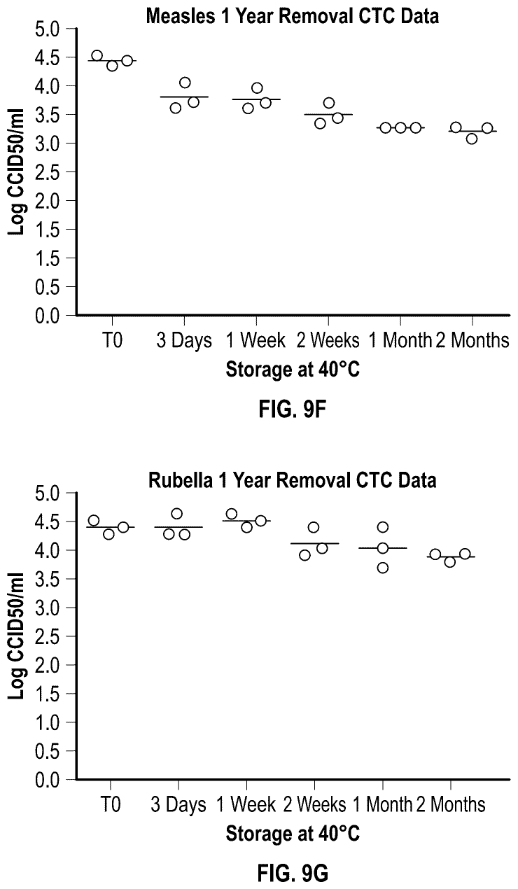

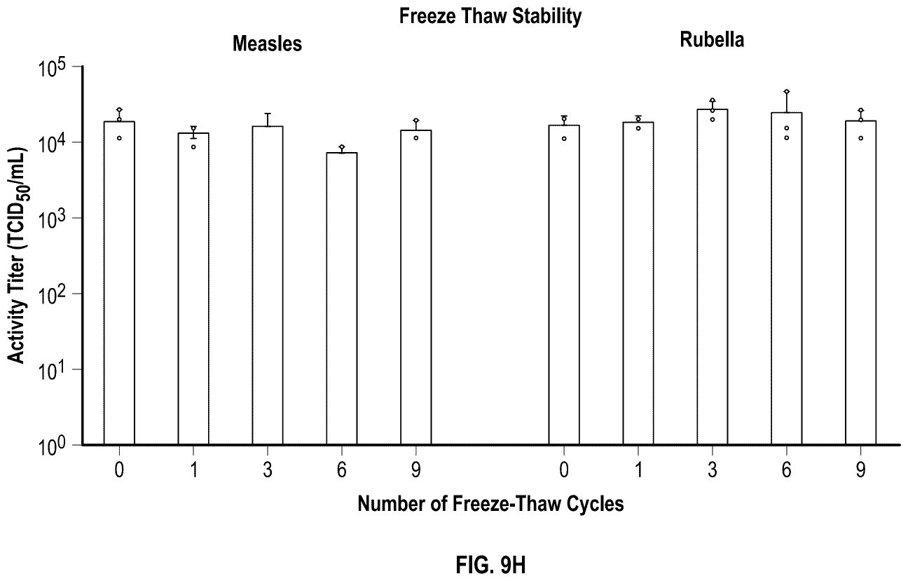

For MRV-MNP samples stored at 5° C.±3° C. (FIG. 9A), the measles (left) and rubella (right) vaccine samples were stable for at least 12 months. For MRV-MNP samples stored at 2° C. to 8° C., the measles (FIG. 9B, left) and the rubella (FIG. 9C, left) vaccine samples were stable for at least 12 months. For MRV-MNP samples stored at 25° C., the measles (FIG. 9B, right) and the rubella (FIG. 9C, right) vaccine samples were stable for at least 12 months. For MRV-MNP samples stored at 37° C., the measles vaccine samples (FIG. 9D) and the rubella vaccine samples (FIG. 7E) were stable for at least 1 month.

For MRV-MNP samples stored under Controlled Temperature Chain (CTC) conditions, i.e., at 2° C. to 8° C. for 12 months, then at 40° C. and 75% relative humidity, the measles vaccine samples (FIG. 9F) and the rubella vaccine samples (FIG. 9G) were stable for at least 2 weeks. At 1 month and 2 months following removal from the 2° C. to 8° C. conditions, the measles and rubella content was still greater than 1000 TCID₅₀, which is the minimum titer required for MR vaccines. FIG. 9H illustrates that measles and rubella vaccines remained stable after being subjected to up to 9 freeze-thaw cycles (i.e., −20° C. for 1 day, followed by 25° C. for 1 day).

MR MAP Thermostability study: 24-month results
Long-termstabilityandVVMlabelingstudydesign: Testcondition,
control, testsamples, andreplicates

| | | Weeks | | Months | | | | | | |
|---|---|---|---|---|---|---|---|---|---|---|
| Conditions | T = 0 | 1 | 2 | 1 | 3 | 6 | 9 | 12 | 18 | 24 |
| 37° C./RH75% | ✓ | ✓ | ✓ | ✓ | ✓ | NT | NT | NT | NT | NT |
| (N = 3) | | | | | | | | | | |
| 25° C./RH65% | | NT | NT* | ✓ | ✓ | ✓ | ✓ | ✓ | ✓ | ✓ |
| (N = 3) | | | | | | | | | | |
| 2-8° C. | | NT | NT | ✓ | ✓ | ✓ | ✓ | ✓ | ✓ | ✓ |
| (N = 3) | | | | | | | | | | |

NT—not tested
V—testing timepoint

CTC set up and time points

Removal

| timepoint | Duration of storage at 40° C. | | | | |
|---|---|---|---|---|---|
| from 2° C.- | 3 | 1 | 2 | 1 | 2 |
| 8° C. storage* | days | week | weeks | month | months |
| @12 months | ✓ | ✓ | ✓ | ✓ | ✓ |
| @24 months | ✓ | ✓ | ✓ | ✓ | ✓ |

*N = 3 MR MAPs were removed and exposed to 40° C. under different storage conditions

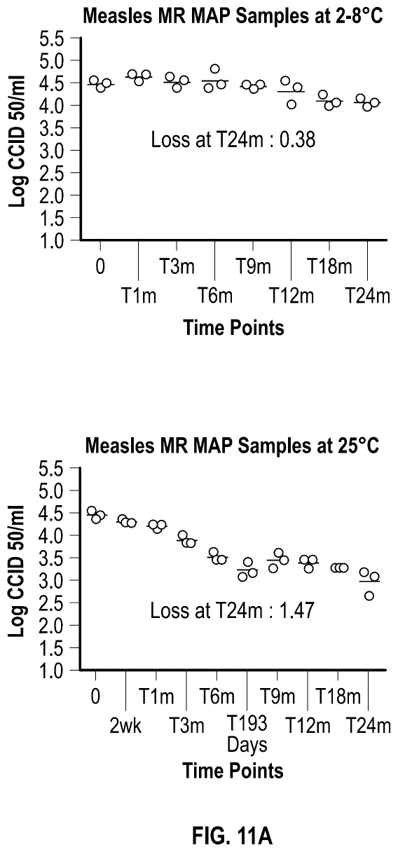

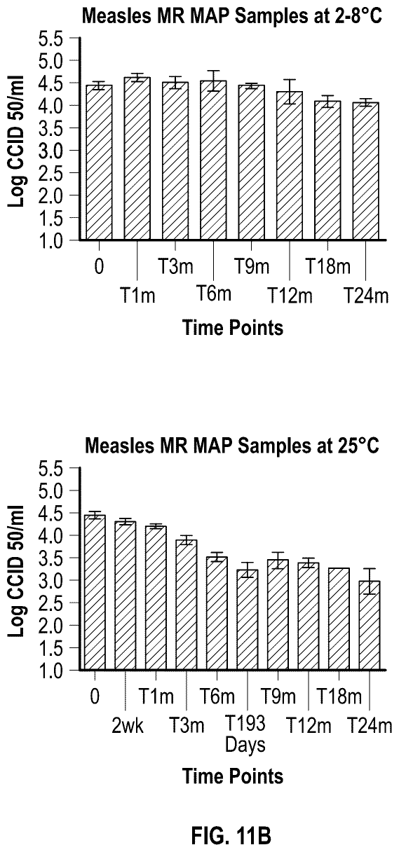

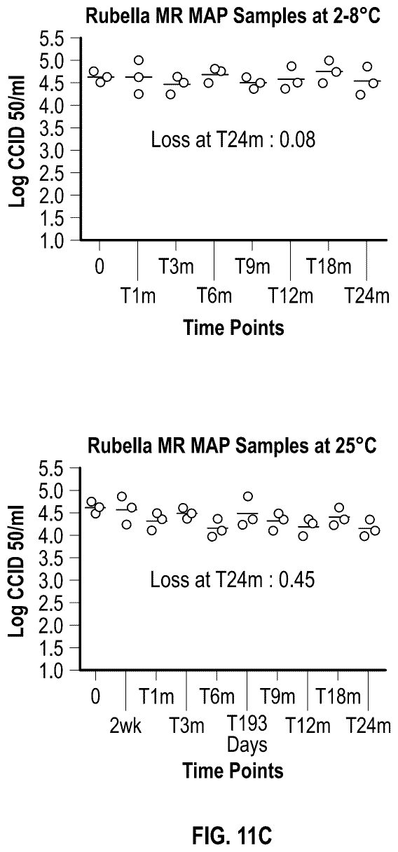

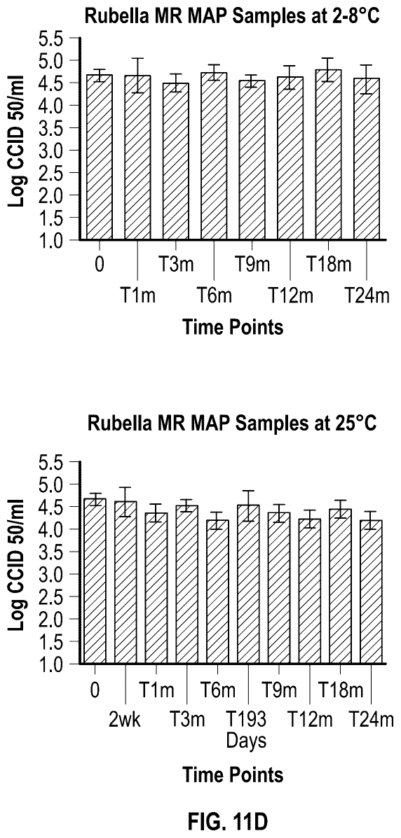

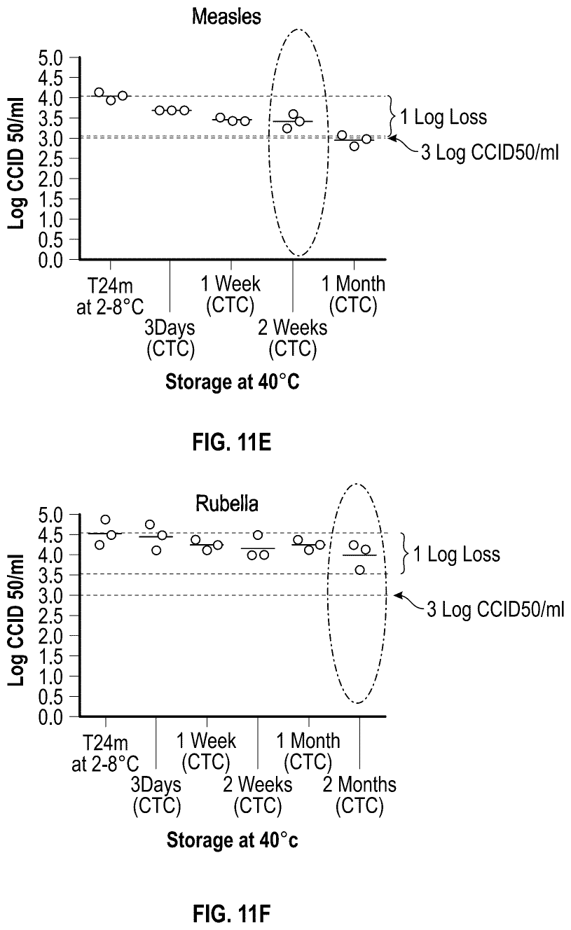

The testing of 24-month stability timepoint for Micron MR MAPs stored at 2° C.-8° C. and 25° C. was completed. The MR MAPs stored at 2° C. to 8° C. continued to maintain Measles and Rubella content with ≤0.5 log loss at the end of 24-month storage. The specific loss for Measles was 0.38 log (FIG. 11A-B) while for Rubella it was 0.08 log loss (FIG. 11C-D) at the end of 24 months at 2-8° C. storage. At the end of 24 months at 25° C. storage, the average loss in Measles content was approximately 1.47 log (FIG. 11A-B) while the average loss in Rubella content was 0.45 log (FIG. 11C-D).

For MRV-MNP samples stored under Controlled Temperature Chain (CTC) conditions, i.e., at 2° C. to 8° C. for 24 months, then at 40° C., the measles vaccine samples (FIG. 11E; Table 9) and the rubella vaccine samples (FIG. 11F, Table 10) were stable. Table 11 provides the average log loss at the different CTC timepoints for measles and rubella vaccines.

TABLE 9
Measles CTC Stability

| Timepoints | Average titer (Log CCID50/ml) |
|---|---|
| T24 m at 2-8° C. | 4.0500 |
| 3 days (CTC) | 3.7000 |
| 1 week (CTC) | 3.4667 |
| 2 weeks (CTC) | 3.4375 |
| 1 month (CTC) | 2.9708 |

TABLE 10
Rubella CTC Stability

| Timepoints | Average titer (Log CCID50/ml) |
|---|---|
| T24 m at 2-8° C. | 4.5417 |
| 3 days (CTC) | 4.4583 |
| 1 week (CTC) | 4.2500 |
| 2 weeks (CTC) | 4.1667 |
| 1 month (CTC) | 4.2500 |
| 2 months (CTC) | 4.0000 |

TABLE 11
Summary

| Time points | | 3 days | 1 week | 2 weeks | 1 month | 2 months |
|---|---|---|---|---|---|---|
| Average | Measles | 0.35 | 0.5833 | 0.6125 | 1.0792 | Below |
| Log loss | | | | | | LOQ |
| | Rubella | 0.0834 | 0.2917 | 0.375 | 0.2917 | 0.5417 |

### Example 7. Acceptability Results

Forty-five adults, 120 measles and rubella vaccine (MR)-primed toddlers, and 120 MR-naïve infants were recruited serially in The Gambia to receive both the microneedle patch (MNP) and subcutaneous injection (SC) in a double dummy design. Acceptability questionnaires were administered to adult participants and parents of toddlers and infants before (pre-vaccination), immediately after (post-vaccination), 14 days after (Day 14) and 42 days after (Day 42) MNP application. Crying data was collected immediately after vaccination. The acceptability of the MNP was high in all cohorts.

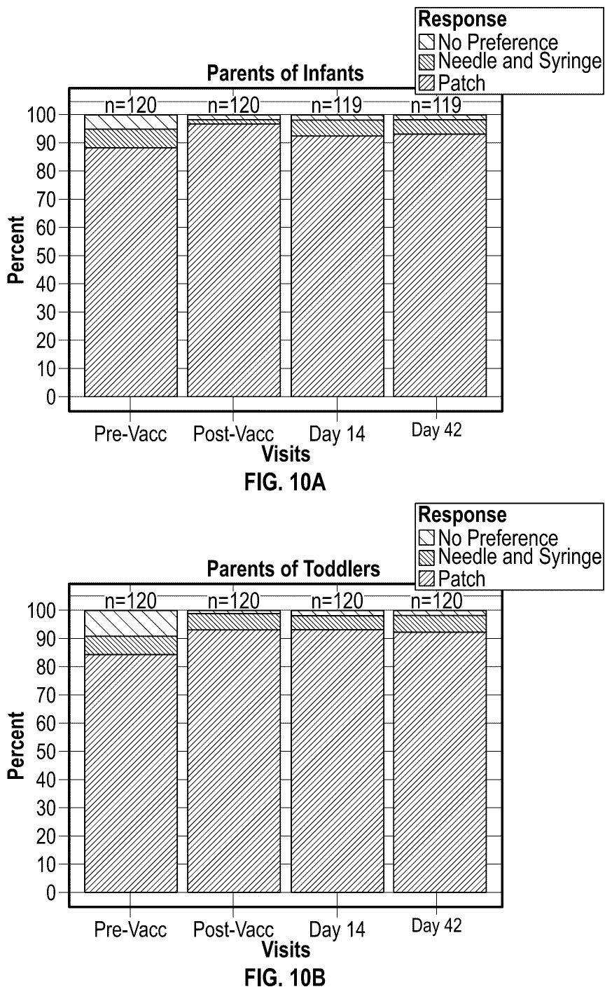

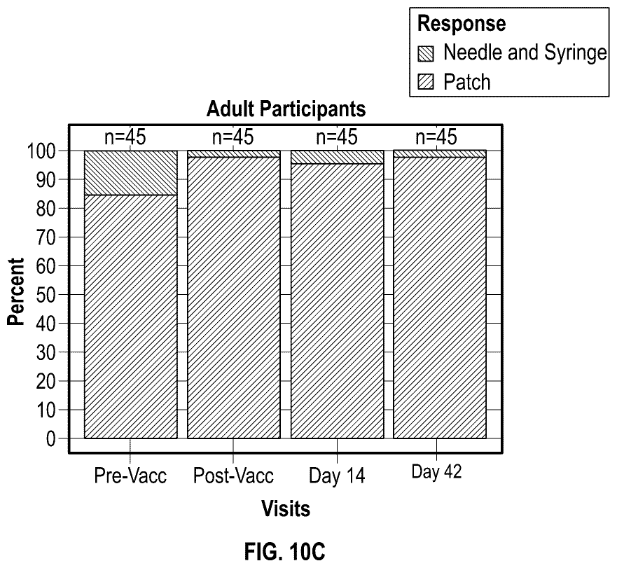

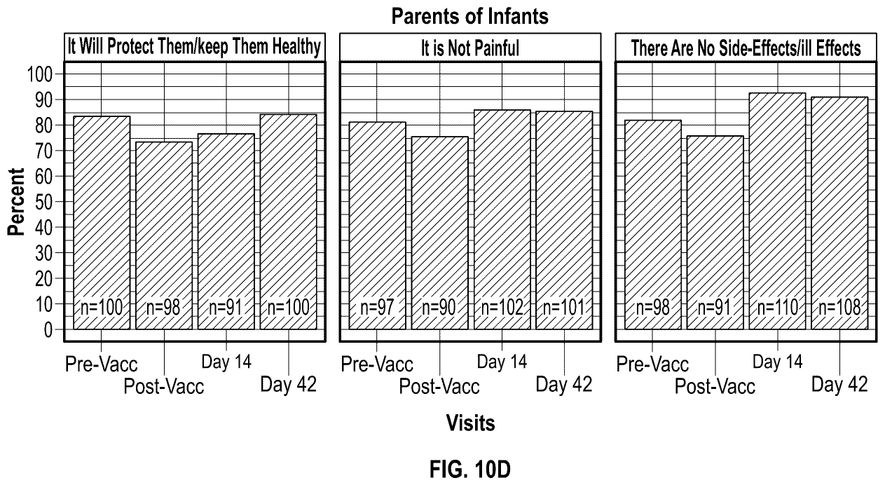

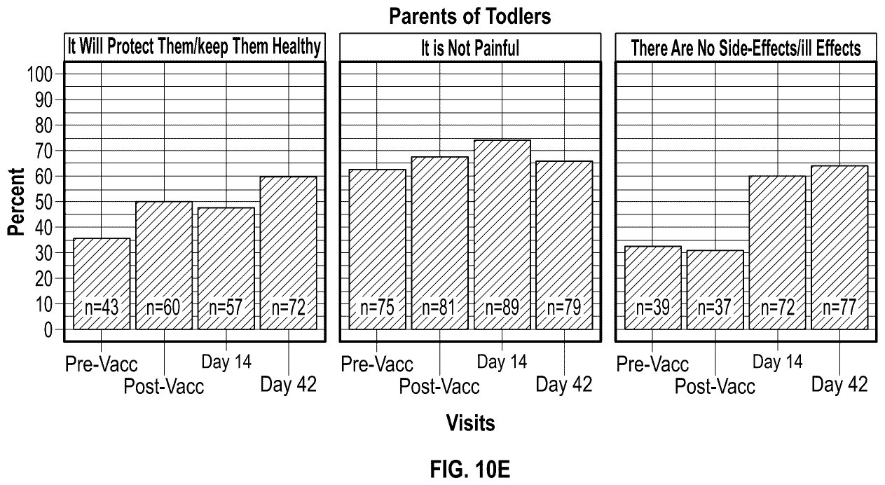

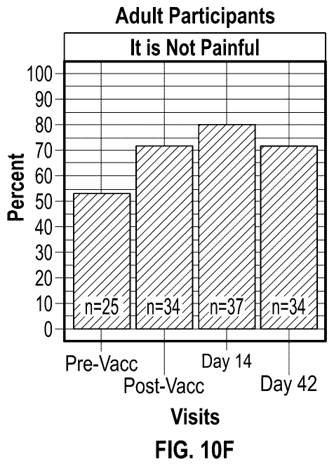

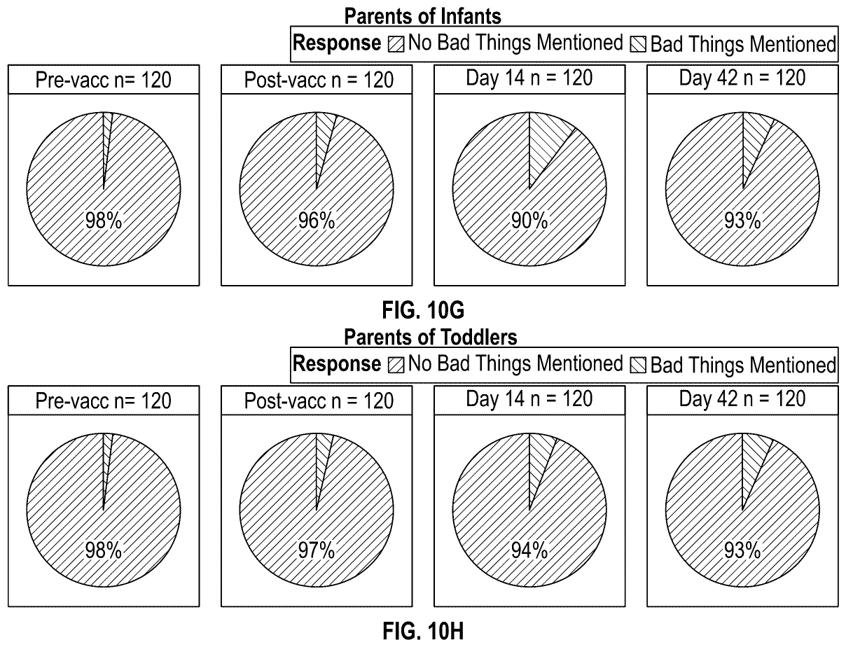

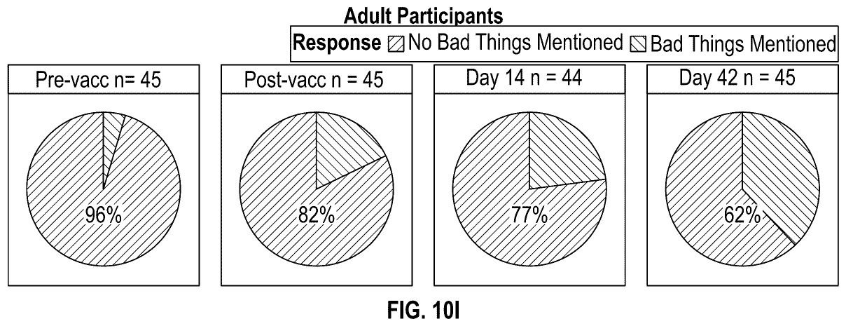

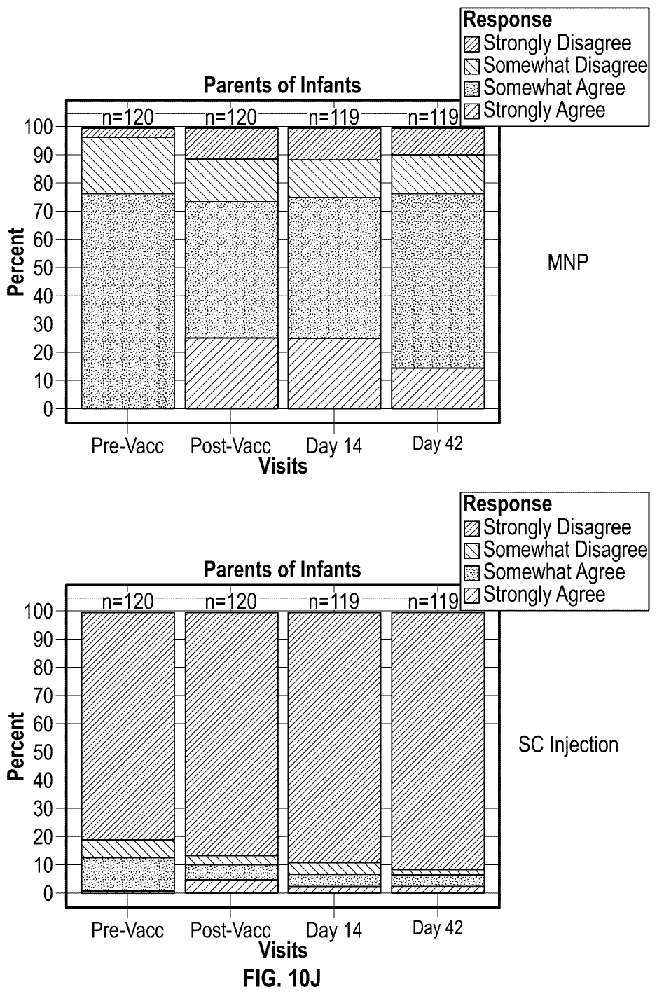

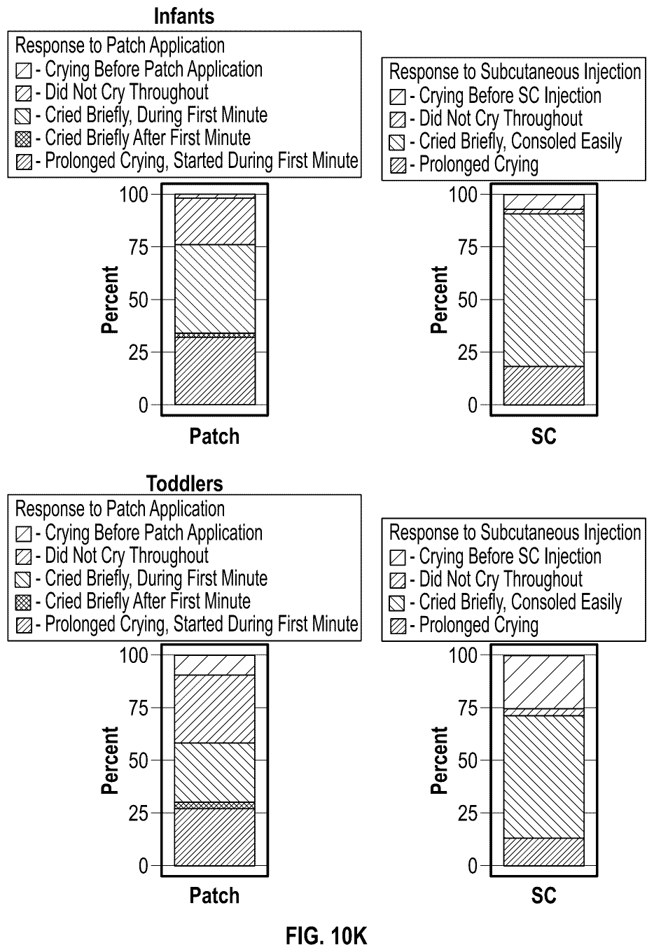

Adult participants and the parents of infants and toddlers who participated in the trial were asked whether MNP or SC injection would be better for administering vaccines to children in The Gambia. Pre-vaccination (i.e., prior to MNP administration), the vast majority of adult participants (79%) and of the parents of infants (89%) and toddlers (86%) said that a MNP would be better than SC. Post-vaccination, i.e. after the adult participants and the parents of participants were able to experience MNP application to themselves (adult participants) or to their children (parents of infants and toddlers), the preference for MNP over SC increased in all three cohorts to 98% (adults), 93% (parents of infants), and 93% (parents of toddlers) by Day 42, further reinforcing the strong preference for MNP over SC at the beginning of the trial. (FIGS. 10A-10C).

When asked about the good things about a child being vaccinated with a patch, the most common answers that the parents of infants and toddlers chose from a list of answers were “it will protect them/keep them healthy”, “it is not painful”, and “there are no side effects/ill effects” (FIGS. 10D-10E). For both groups and for all three most common answers, the percentage of parents who selected these answers increased from pre-vaccination to Day 42, indicating that the parent's positive view of MNP increased as they were exposed to MNP vaccination during the trial. For adult participants, the most common answer was “it is not painful” (FIG. 10F).

When asked about the bad things about a child being vaccinated with a patch, the vast majority of parents of infant and toddler participants (98% pre-vaccination and 93% at Day 42) and the majority of adult participants (92% pre-vaccination and 62% at Day 42) reported no bad things (FIGS. 10G-10I).

When parents of infants were asked about their comfort level with volunteers, who had never previously used an MNP, administering a MNP vaccine to their child after a short training, the vast majority of parents (77% pre-vaccination and 77% at Day 42) said they strongly or somewhat agreed that they would be comfortable. When asked the same question but with volunteers using a hypodermic needles and syringe (SC injection) if they had never used a needle and syringe before, to vaccinate their child, the vast majority of parents (86% pre-vaccination and 94% at Day 42) strongly or somewhat disagreed that they would feel comfortable (FIG. 10J).

Results regarding crying when infants (FIG. 10K) and toddlers (FIG. 10K) were given a vaccine via a MNP (left) and subcutaneous injection (right) show that for infants and toddlers, more children cried during vaccination if vaccination was performed by SC compared to MNP vaccination. Also, for both infants and toddlers, the percentage of children who did not cry at all was much higher if vaccination was performed by MNP compared to SC injection.

### EMBODIMENTS

Some embodiments of the present disclosure can be described in view of one or more of the following:

Embodiment 1. A method for vaccinating a child or infant, the method comprising: applying to an area of skin of the child or infant a drug delivery device comprising an array of microneedles comprising a vaccine formulation which comprises at least one infectious disease antigen, wherein the applying is effective to administer the infectious disease antigen to the child or infant via the array of microneedles and to protect the child or infant from an infectious disease corresponding with the infectious disease antigen.

Embodiment 2. The method of Embodiment 1, wherein the method is effective to ameliorate trypanophobia in the child or in a parent or other caretaker of the child or infant.

Embodiment 3. The method of Embodiment 1 or 2, wherein the infectious disease antigen is a live virus antigen.

Embodiment 4. The method of Embodiment 1 or 2, wherein the infectious disease antigen is an attenuated live virus antigen.

Embodiment 5. The method of any one of Embodiments 1 to 4, wherein the microneedles are dissolvable microneedles.

Embodiment 6. The method of Embodiment 5, wherein at least 60%, at least 75% or at least 90%, of the height of the dissolvable microneedles dissolves into the skin of the infant or child, optionally wherein the dissolution alters the tip geometries of the microneedles, whereby the microneedles are no longer able to penetrate the stratum corneum of skin.

Embodiment 7. The method of any one of Embodiments 1 to 4, wherein the array of microneedles are microneedles each having a coating of the vaccine formulation thereon.

Embodiment 8. The method of any one of Embodiments 1 to 7, wherein a reaction to the vaccine in the child or the infant comprises a mild local reaction in the area of skin, the reaction initially occurring within 14 days of the applying of the drug delivery device.

Embodiment 9. The method of Embodiment 8, wherein the reaction to the vaccine in the child or the infant consists essentially of a mild local reaction in the area of the skin, the reaction initially occurring within 14 days of the applying of the drug delivery device.

Embodiment 10. The method of Embodiment 8 or 9, wherein the mild local reaction in the area of the skin is indicative of an effective vaccination, optionally persisting beyond 14 days of the applying of the drug delivery device.

Embodiment 11. The method of any one of Embodiments 1 to 10, wherein the child or infant was previously vaccinated before the applying of the drug delivery device.

Embodiment 12. The method of Embodiment 11, wherein the child or infant received a prior vaccination to the same infectious disease antigen as that of the vaccine formulation.

Embodiment 13. The method of any one of Embodiments 1 to 10, wherein the vaccine formulation administered by the drug delivery device is the first vaccination of the infectious disease antigen administered to the child or infant.

Embodiment 14. The method of any one of Embodiments 1 to 13, wherein an induration response in the child or infant occurs earlier in children or infants with previous exposure to the infectious disease antigen than in children or infants without previous exposure to the infectious disease antigen.

Embodiment 15. The method of any one of Embodiments 1 to 14, wherein the vaccine formulation comprises at least two different infectious disease antigens.

Embodiment 16. The method of any one of Embodiments 1 to 15, wherein the drug delivery device is a patch applied to the skin, preferably of an arm or wrist, of the infant or child, and the infant or child tolerates the application of the patch for the period of application of the patch.

Embodiment 17. The method of Embodiment 16, wherein the period of the application of the patch is 5 minutes or less, preferably 1 minute or less, or more preferably 10 seconds or less.

Embodiment 18. The method of any one of Embodiments 1 to 17, wherein the protection of the child or infant from an infectious disease is demonstrated by a seroconversion of the child or infant to the infectious disease antigen delivered by the microneedles that is statistically equivalent to or greater than the seroconversion of the infectious disease antigen delivered by subcutaneous or intramuscular injection.

Embodiment 19. The method of any one of Embodiments 1 to 17, wherein the protection of the child or infant from an infectious disease is demonstrated by a serum neutralizing antibody seroprotection of the child to the infectious disease antigen delivered by the microneedles that is statistically equivalent to the serum neutralizing antibody seroprotection of the infectious disease antigen delivered by subcutaneous injection at 42 days post vaccination.

Embodiment 20. The method of Embodiment 19, wherein the seroprotection rate is statistically equivalent for at least 180 days post vaccination.

Embodiment 21. The method of any one of Embodiments 1 to 20, wherein the infectious disease is selected from the group consisting of influenza, COVID-19, measles, diphtheria, tetanus, pertussis, dengue, hepatitis A, hepatitis B, mumps, human papilloma virus (HPV), pneumococcal, meningococcal, rotavirus, polio, varicella, rubella, smallpox, monkeypox, and combinations thereof.

Embodiment 22. The method of any one of Embodiments 1 to 21, wherein the vaccine formulation is thermostable in the drug delivery device for at least one of 1 month at 37° C., 12 months at 25° C., or 12 months at 5° C.

Embodiment 23. The method of Embodiment 22, wherein the vaccine formulation is thermostable in the drug delivery device for at least 3 months, optionally up to 12 months, at 5° C. followed by 14 days at 40° C./75% RH.

Embodiment 24. The method of any one of Embodiments 1 to 23, wherein the infectious disease antigen administered is a fractional dose.

Embodiment 25. The method of any one of Embodiments 1 to 24, wherein the child or infant is a child from four years old to six years old.

Embodiment 26. The method of any one of Embodiments 1 to 24, wherein the child or infant is an infant from four months old to eighteen months old.

Embodiment 27. The method of any one of Embodiments 1 to 26, wherein the drug delivery device is self-administered or applied by an individual who is not a trained medical professional.

Embodiment 28. The method of any one of Embodiments 1 to 27, wherein the drug delivery device further comprises a force feedback indicator (FFI) and wherein the drug delivery device is applied manually to the skin such that the FFI provides at least one of an audible, tactile, or visual indication that the microneedles have been inserted in the skin.

Embodiment 29. The method of Embodiment 28, wherein the FFI provides audible, tactile, and visual indications that a button positioned over the microneedle array has been fully depressed within a housing of the drug delivery device.

Embodiment 30. The method of any one of Embodiments 1 to 29, which produces hyperpigmentation of the skin of the infant or child effective to visually indicate that the infant or child has received the vaccination.

Embodiment 31. The method of any one of Embodiments 1 to 30, wherein, before the drug delivery device is applied to the area of skin of the child or infant, the drug delivery device optionally may have been transported or stored outside of cold chain conditions for more than 12 hours or for at least 14 days.

Embodiment 32. A method of ameliorating trypanophobia in an individual in need of vaccination comprising: administering a vaccine to the individual by applying to an area of skin of the individual a drug delivery device having an array of microneedles comprising a vaccine formulation which comprises an infectious disease antigen.

Embodiment 33. The method of Embodiment 32, wherein the individual is a child or adult.

Embodiment 34. The method of Embodiment 32 or 33, wherein the array of microneedles are dissolvable microneedles.

Embodiment 35. The method of Embodiment 34, wherein at least 50%, at least 60%, at least 75% or at least 90%, of the height of the dissolvable microneedles dissolves into the skin of the individual, optionally wherein the dissolution alters the tip geometries of the microneedles, whereby the microneedles are no longer able to penetrate the stratum corneum of skin.

Embodiment 36. The method of Embodiment 32 or 33, wherein the array of microneedles are microneedles each having a coating of the vaccine formulation thereon.

Embodiment 37. A drug delivery device configured for use in the methods of any one of Embodiments 1 to 36.

Embodiment 38. The drug delivery device of Embodiment 37, wherein the drug delivery device is a unit of a single dose of the vaccine, optionally packaged as a kit, or within a box, comprising a plurality of said units.

Embodiment 39. The drug delivery device of Embodiment 37 or 38, wherein the drug delivery device is sterile, or has a low bioburden.

Embodiment 40. The method of any of Embodiments 1 to 36, wherein the application of the array of microneedles is better tolerated by the infant or child than a subcutaneous injection to administer the at least one infectious disease antigen.

Embodiment 41. A method for vaccinating an adult, the method comprising: applying to an area of skin of the adult a drug delivery device having an array of microneedles comprising a vaccine formulation which comprises at least one infectious disease antigen, wherein the applying is effective to administer the infectious disease antigen to the adult via the array of microneedles and to protect the adult from an infectious disease corresponding with the infectious disease antigen, and wherein the applying is effective to protect the adult from the infectious disease even if, prior to the applying step, the drug delivery device is transported or stored out of cold chain conditions for at least 12 hours, such as for up to 14 days.

Embodiment 42. The method of Embodiment 41, wherein the method is effective to ameliorate trypanophobia in the adult.

Embodiment 43. The method of Embodiment 41 or 42, wherein the infectious disease antigen is a live virus antigen.

Embodiment 44. The method of Embodiment 41 or 42, wherein the infectious disease antigen is an attenuated live virus antigen.

Embodiment 45. The method of any one of Embodiments 41 to 44, wherein the microneedles are dissolvable microneedles.

Embodiment 46. The method of Embodiment 45, wherein at least 50%, at least 60%, at least 75% or at least 90%, of the height of the dissolvable microneedles dissolves into the skin of the adult, optionally wherein the dissolution alters the tip geometries of the microneedles, whereby the microneedles are no longer able to penetrate the stratum corneum of skin.

Embodiment 47. The method of any one of Embodiments 41 to 44, wherein the array of microneedles are microneedles each having a coating of the vaccine formulation thereon.

Embodiment 48. The method of any one of Embodiments 41 to 47, wherein a reaction to the vaccine in the adult comprises a mild local reaction in the area of skin, the reaction occurring within 14 days of the applying of the drug delivery device.

Embodiment 49. The method of Embodiment 48, wherein the reaction to the vaccine in the adult consists essentially of a mild local reaction in the area of the skin, the reaction occurring within 14 days of the applying of the drug delivery device.

Embodiment 50. The method of Embodiment 49, wherein the mild local reaction in the area of the skin is indicative of an effective vaccination.

Embodiment 51. The method of any one of Embodiments 42 to 50, wherein the adult was previously vaccinated before the applying of the drug delivery device.

Embodiment 52. The method of Embodiment 51, wherein the adult received a prior vaccination to the same infectious disease antigen as that of the vaccine formulation.

Embodiment 53. The method of any one of Embodiments 42 to 50, wherein the vaccine formulation administered by the drug delivery device is the first vaccination of the infectious disease antigen administered to the adult.

Embodiment 54. The method of any one of Embodiments 42 to 53, wherein an induration response in the adult occurs earlier in adults with previous exposure to the infectious disease antigen than in adults without previous exposure to the infectious disease antigen.

Embodiment 55. The method of any one of Embodiments 42 to 54, wherein the vaccine formulation comprises at least two different infectious disease antigens.

Embodiment 56. The method of any one of Embodiments 42 to 55, wherein the drug delivery device is a patch applied to the skin, preferably of an arm or wrist, of the adult, and the adult tolerates the application of the patch for the period of application of the patch.

Embodiment 57. The method of Embodiment 56, wherein the period of the application of the patch is 5 minutes or less, preferably 1 minute or less, or more preferably 10 seconds or less.

Embodiment 58. The method of any one of Embodiments 42 to 57, wherein the protection of the adult from an infectious disease is demonstrated by a seroconversion of the adult to the infectious disease antigen delivered by the microneedles that is statistically equivalent to or greater than the seroconversion of the infectious disease antigen delivered by subcutaneous or intramuscular injection.

Embodiment 59. The method of any one of Embodiments 42 to 57, wherein the protection of the adult from an infectious disease is demonstrated by a serum neutralizing antibody seroprotection of the adult to the infectious disease antigen delivered by the microneedles that is statistically equivalent to the serum neutralizing antibody seroprotection of the infectious disease antigen delivered by subcutaneous injection at 42 days post vaccination.

Embodiment 60. The method of Embodiment 49, wherein the seroprotection rate is statistically equivalent for at least 180 days post vaccination.

Embodiment 61. The method of any one of Embodiments 42 to 50, wherein the infectious disease is selected from the group consisting of influenza, COVID-19, measles, diphtheria, tetanus, pertussis, dengue, hepatitis A, hepatitis B, mumps, human papilloma virus (HPV), pneumococcal, meningococcal, rotavirus, polio, varicella, rubella, smallpox, monkeypox and combinations thereof.

Embodiment 62. The method of any one of Embodiments 42 to 61, wherein the vaccine formulation is thermostable in the drug delivery device for at least one of 1 month at 37° C., 12 months at 25° C., or 12 months at 5° C.

Embodiment 63. The method of Embodiment 62, wherein the vaccine formulation is thermostable in the drug delivery device for at least 3 months, optionally up to 12 months, at 5° C. followed by 14 days at 40° C./75% RH.

Embodiment 64. The method of any one of Embodiments 42 to 63, wherein the infectious disease antigen administered is a fractional dose.

Embodiment 65. The method of any one of Embodiments 42 to 64, wherein the adult is from 18 years old to 40 years old.

Embodiment 66. The method of any one of Embodiments 42 to 65, wherein the drug delivery device is self-administered or applied by an individual who is not a medical professional.

Embodiment 67. The method of any one of Embodiments 42 to 66, wherein the drug delivery device further comprises a force feedback indicator (FFI) and wherein the drug delivery device is applied manually to the skin such that the FFI provides at least one of an audible, tactile, or visual indication that the microneedles have been inserted in the skin.

Embodiment 68. The method of Embodiment 67, wherein the FFI provides audible, tactile, and visual indications that a button positioned over the microneedle array has been fully depressed within a housing of the drug delivery device.

Embodiment 69. The method of any one of Embodiments 42 to 68, which produces hyperpigmentation of the skin of the adult effective to visually indicate that the adult has received the vaccination.

Embodiment 70. The method of any one of Embodiments 1 to 69, wherein, before the drug delivery device is applied to the area of skin of the adult, the drug delivery device has been transported or stored outside of cold chain conditions for more than 12 hours or for at least 14 days.

Embodiment 71. A method for vaccinating a subject, the method comprising: applying to an area of skin of the subject, for an application period, a drug delivery device comprising an array of dissolvable microneedles comprising a vaccine formulation which comprises at least one infectious disease antigen, wherein: the applying is effective to penetrate the stratum corneum of the skin with the array of dissolvable microneedles and to administer the at least one infectious disease antigen to the subject, and at least 60% of the height of the dissolvable microneedle dissolves into the skin of the subject.

Embodiment 72. The method of Embodiment 71, wherein the applying is effective to protect the subject from at least one infectious disease corresponding with the at least one infectious disease antigen.

Embodiment 73. The method of Embodiment 71 or 72, wherein at least 70% of the height of the dissolvable microneedle dissolves into the skin of the subject.

Embodiment 74. The method of Embodiment 71 or 72, wherein at least 75% of the height of the dissolvable microneedle dissolves into the skin of the subject.

Embodiment 75. The method of Embodiment 71 or 72, wherein at least 80% of the height of the dissolvable microneedle dissolves into the skin of the subject.

Embodiment 76. The method of Embodiment 71 or 72, wherein at least 85% of the height of the dissolvable microneedle dissolves into the skin of the subject.

Embodiment 77. The method of Embodiment 71 or 72, wherein at least 90% of the height of the dissolvable microneedle dissolves into the skin of the subject.

Embodiment 78. The method of any one of Embodiments 71 to 77, wherein the dissolution alters the geometry of the tip of each microneedles such that it is no longer able to penetrate the stratum corneum of skin.

Embodiment 79. The method of any one of Embodiments 71 to 78, wherein the infectious disease antigen is a live virus antigen.

Embodiment 80. The method of any one of Embodiments 71 to 78, wherein the infectious disease antigen is an attenuated live virus antigen.

Embodiment 81. The method of any one of Embodiments 71 to 80, wherein the drug delivery device is a patch applied to the skin, preferably of an arm or wrist, of the subject.

Embodiment 82. The method of Embodiment 81, wherein the subject tolerates the application of the patch for the application period.

Embodiment 83. The method of Embodiment 81 or 82, wherein the application period is 5 minutes or less, preferably 1 minute or less, or more preferably 10 seconds or less.

Embodiment 84. The method of any one of Embodiments 71 to 83, wherein the protection of the subject from an infectious disease is demonstrated by a seroconversion of the subject to the infectious disease antigen delivered by the microneedles that is statistically equivalent to or greater than the seroconversion of the infectious disease antigen delivered by subcutaneous or intramuscular injection.

Embodiment 85. The method of any one of Embodiments 71 to 84, wherein the protection of the subject from an infectious disease is demonstrated by a serum neutralizing antibody seroprotection of the subject to the infectious disease antigen delivered by the microneedles that is statistically equivalent to the serum neutralizing antibody seroprotection of the infectious disease antigen delivered by subcutaneous injection at 42 days post vaccination.

Embodiment 86. The method of Embodiment 85, wherein the seroprotection rate is statistically equivalent for at least 180 days post vaccination.

Embodiment 87. The method of any one of Embodiments 71 to 86, wherein the infectious disease is selected from the group consisting of influenza, COVID-19, measles, diphtheria, tetanus, pertussis, dengue, hepatitis A, hepatitis B, mumps, human papilloma virus (HPV), pneumococcal, meningococcal, rotavirus, polio, varicella, rubella, smallpox, monkeypox, and combinations thereof.

Embodiment 88. The method of any one of Embodiments 71 to 87, wherein the subject is an infant.

Embodiment 89. The method of Embodiment 81, wherein the infant is from four months old to eighteen months old.

Embodiment 90. The method of any one of Embodiments 71 to 87, wherein the subject is a child.

Embodiment 91. The method of Embodiment 90, wherein the child is from four years old to six years old.

Embodiment 92. The method of any one of Embodiments 71 to 87, wherein the subject is an adult.

Embodiment 93. The method of Embodiment 92, wherein the adult is from 18 years old to 40 years old.

Embodiment 94. The method of any one of Embodiments 71 to 87, wherein the drug delivery device is self-administered or applied by an individual who is not a medical professional.

Embodiment 95. A device comprising an array of dissolvable microneedles, optionally configured for use in any of the methods of Embodiments 1 to 94.

Embodiment 96. The device of Embodiment 95, wherein the vaccine formulation further comprises gelatin, sorbitol, and one or more buffers.

Embodiment 97. The device of Embodiment 95 or 96, wherein the at least one infectious disease antigen comprises a measles antigen and a rubella antigen.

Embodiment 98. The device of any one of Embodiment 95 to 97, wherein the at least one infectious disease antigen comprises a measles antigen, a mumps antigen, and a rubella antigen.

That which is claimed is:

1. A method for vaccinating a child or infant, the method comprising:
applying to an area of skin of the child or infant a drug delivery device comprising an array of microneedles comprising a vaccine formulation which comprises at least one infectious disease antigen,
wherein the applying is effective to administer the infectious disease antigen to the child or infant via the array of microneedles and to protect the child or infant from an infectious disease corresponding with the at least one infectious disease antigen.

2. The method of claim 1, wherein the infectious disease antigen is a live virus antigen.

3. The method of claim 1, wherein the infectious disease antigen is an attenuated live virus antigen.

4. The method of claim 1, wherein the microneedles are dissolvable microneedles, and at least 50% preferably at least 75% or more preferably at least 90%, of the height of the dissolvable microneedles dissolves into the skin of the infant or child, optionally wherein the dissolution alters the tip geometries of the microneedles, whereby the microneedles are no longer able to penetrate the skin.

5. The method of claim 1, wherein the array of microneedles are microneedles each having a coating of the vaccine formulation thereon.

6. The method of claim 1, wherein a reaction to the vaccine in the child or the infant comprises a mild local reaction in the area of skin, the reaction initially occurring within 14 days of the applying of the drug delivery device.

7. The method of claim 1, wherein a reaction to the vaccine in the child or the infant consists of a mild local reaction in the area of the skin, the reaction initially occurring within 14 days of the applying of the drug delivery device.

8. The method of claim 6, wherein the mild local reaction in the area of the skin is indicative of an effective vaccination, optionally persisting beyond 14 days of the applying of the drug delivery device.

9. The method of claim 1, wherein the vaccine formulation administered by the drug delivery device is the first vaccination of the infectious disease antigen administered to the child or infant.

10. The method of claim 1, wherein the vaccine formulation comprises at least two different infectious disease antigens.

11. The method of claim 1, wherein the drug delivery device is a microneedle patch applied to the skin, preferably of an arm or wrist, of the infant or child, and the infant or child tolerates the application of the patch for the period of application of the patch.

12. The method of claim 11, wherein the period of the application of the microneedle patch is 5 minutes or less.

13. The method of claim 11, wherein the period of the application of the microneedle patch is 1 minute or less.

14. The method of claim 1, wherein the protection of the child or infant from an infectious disease is demonstrated by a seroconversion of the child or infant to the infectious disease antigen delivered by the microneedles that is statistically equivalent to or greater than the seroconversion of the infectious disease antigen delivered by subcutaneous or intramuscular injection.

15. The method of claim 1, wherein the protection of the child or infant from an infectious disease is demonstrated by a serum neutralizing antibody seroprotection of the child to the infectious disease antigen delivered by the microneedles that is statistically equivalent to the serum neutralizing antibody seroprotection of the infectious disease antigen delivered by subcutaneous injection at 42 days post vaccination.

16. The method of claim 15, wherein the seroprotection rate is statistically equivalent for at least 180 days post vaccination.

17. The method of claim 1, wherein the infectious disease is selected from the group consisting of influenza, COVID-19, measles, diphtheria, tetanus, pertussis, dengue, hepatitis A, hepatitis B, mumps, human papilloma virus (HPV), pneumococcal, meningococcal, rotavirus, polio, varicella, rubella, smallpox, monkeypox, and combinations thereof.

18. The method of claim 1, wherein the vaccine formulation is thermostable in the drug delivery device for at least one of 1 month at 37° C., 12 months at 25° C., or 12 months at 5° C.

19. The method of claim 18, wherein the vaccine formulation is thermostable in the drug delivery device for at least 3 months, optionally up to 12 months, at 5° C. followed by 14 days at 40° C.

20. The method of claim 1, wherein, before the drug delivery device is applied to the area of skin of the child or infant, the drug delivery device optionally may have been transported or stored outside of cold chain conditions for more than 12 hours or for at least 14 days.

21. A drug delivery device configured for use in the method of claim 1, wherein the vaccine formulation further comprises gelatin, sorbitol, and one or more buffers.

22. The drug delivery device of claim 21, wherein the at least one infectious disease antigen comprises a measles antigen and a rubella antigen.

23. A method for vaccinating a subject, the method comprising:
applying to an area of skin of the subject, for an application period of five (5) minutes or less, a device comprising an array of dissolvable microneedles comprising a vaccine formulation which comprises at least one infectious disease antigen,
wherein:
the applying is effective to penetrate the stratum corneum of the skin with the array of dissolvable microneedles and to administer the at least one infectious disease antigen to the subject, and
at least 50% of the height of the dissolvable microneedle dissolves into the skin of the subject during the application period.

24. The method of claim 23, wherein at least 70% of the height of the dissolvable microneedle dissolves into the skin of the subject during the application period.

25. The method of claim 23, wherein at least 80% of the height of the dissolvable microneedle dissolves into the skin of the subject during the application period.

26. The method of claim 23, wherein at least 90% of the height of the dissolvable microneedle dissolves into the skin of the subject during the application period.

27. The method of claim 23, wherein the dissolution alters the geometry of the tip of each microneedles such that it is no longer able to penetrate the stratum corneum of skin.

28. The method of claim 23, wherein the infectious disease antigen is a live virus antigen.

29. The method of claim 23, wherein the infectious disease antigen is an attenuated live virus antigen.

30. The method of claim 23, wherein the application period is 1 minute or less, for example between 10 seconds and 30 seconds.

31. The method of claim 23, wherein the protection of the subject from an infectious disease is demonstrated by a seroconversion of the subject to the infectious disease antigen delivered by the microneedles that is statistically equivalent to or greater than the seroconversion of the infectious disease antigen delivered by subcutaneous or intramuscular injection.

32. The method of claim 23, wherein the protection of the subject from an infectious disease is demonstrated by a serum neutralizing antibody seroprotection of the subject to the infectious disease antigen delivered by the microneedles that is statistically equivalent to the serum neutralizing antibody seroprotection of the infectious disease antigen delivered by subcutaneous injection at 42 days post vaccination.

33. The method of claim 32, wherein the seroprotection rate is statistically equivalent for at least 180 days post vaccination.

34. The method of claim 23, wherein the infectious disease is selected from the group consisting of influenza, COVID-19, measles, diphtheria, tetanus, pertussis, dengue, hepatitis A, hepatitis B, mumps, human papilloma virus (HPV), pneumococcal, meningococcal, rotavirus, polio, varicella, rubella, smallpox, monkeypox, and combinations thereof.

35. The method of claim 23, wherein the subject is an infant or child.

36. The method of claim 23, wherein the subject is an adult.

37. The method of claim 23, wherein the drug delivery device is self-administered or applied by an individual who is not a medical professional.

38. A device configured for use in the method of claim 23, wherein the vaccine formulation further comprises gelatin, sorbitol, and one or more buffers.

39. The device of claim 38, wherein the at least one infectious disease antigen comprises a measles antigen and a rubella antigen.
